SEQUENCE LISTING (52 sequences)

SEQ ID NO: 1 - protein, 462 aa, Homo sapiens [sequence omitted]

SEQ ID NO: 2 - protein, 448 aa, Homo sapiens [sequence omitted]

SEQ ID NO: 3 - protein, 454 aa, Homo sapiens
MATNKERLFAAGALGPGSGYPGAGFPFAFPGALRGSPPFEMLSPSFRGLGQPDLPKEMASLSVETQSTSSEEMVPSSPSPPPPPRVYKPCFVCNDKSSGYHYGVSSCEGCKGFFRRSIQKNMVYTCHRDKNCIINKVTRNRCQYCRLQKCFEVGMSKEAVRNDRNKKKKEVKEEGSPDSYELSPQLEELITKVSKAHQETFPSLCQLGKYTTNSSADHRVQLDLGLWDKFSELATKCIIKIVEFAKRLPGFTGLSIADQITLLKAACLDILMLRICTRYTPEQDTMTFSDGLTLNRTQMHNAGFGPLTDLVFAFAGQLLPLEMDDTETGLLSAICLICGDRMDLEEPEKVDKLQEPLLEALRLYARRRRPSQPYMFPRMLMKITDLRGISTKGAERAITLKMEIPGPMPPLIREMLENPEMFEDDSSQPGPHPNASSEDEVPGGQGKGGLKSPA

SEQ ID NO: 4 - protein, 9 aa, Artificial Sequence
  synthetic peptide
LXXIIXXXL

SEQ ID NO: 5 - protein, 8 aa, Artificial Sequence
  synthetic peptide
DYKDDDDK

SEQ ID NO: 6 - protein, 9 aa, Artificial Sequence
  synthetic peptide
YPYDVPDYA

SEQ ID NO: 7 - protein, 10 aa, Artificial Sequence
  synthetic peptide
EQKLISEEDL

SEQ ID NO: 8 - protein, 6 aa, Artificial Sequence
  synthetic peptide
DTYRYI

SEQ ID NO: 9 - protein, 6 aa, Artificial Sequence
  synthetic peptide
HHHHHH

SEQ ID NO: 10 - DNA, 17 bp, Artificial Sequence
  synthetic nucleotide
ggttcannnnnagttca

SEQ ID NO: 11 - DNA, 14 bp, Artificial Sequence
  synthetic nucleotide
aggtcannaggtca

SEQ ID NO: 12 - DNA, 35 bp, Artificial Sequence
  synthetic nucleotide
aggtgannnaggttannnnnnnnnnnnnngggtca

SEQ ID NO: 13 - DNA, 13 bp, Artificial Sequence
  synthetic nucleotide
agggcanaggtca

SEQ ID NO: 14 - DNA, 13 bp, Artificial Sequence
  synthetic nucleotide
cggcaanaggtca

SEQ ID NO: 15 - DNA, 15 bp, Artificial Sequence
  synthetic nucleotide
gggtcannnggttca

SEQ ID NO: 16 - DNA, 15 bp, Artificial Sequence
  synthetic nucleotide
agaacannntgttct

SEQ ID NO: 17 - DNA, 27 bp, Artificial Sequence
  synthetic nucleotide
agctttcaggtcaccaggaggtcagaa

SEQ ID NO: 18 - protein, 57 aa, Homo sapiens
  VARIANT (1)...(57): Xaa = Any Amino Acid
GKHYGVYSCEGCKGFFKRTVRXDLTYTCRDNKDCLIDKRQRNRCQYCRYQKCLAMGM

SEQ ID NO: 19 - protein, 57 aa, T. cystophoro
VKHYGVFACEQCKGFFKRSVRNNRKYSCLGKRHCDTDKKSRNRCQYCRFQKCVQVGM

SEQ ID NO: 20 - protein, 83 aa, Homo sapiens
GYHYRCITCEQCKQFFRRTTQKNLHPSYSCKYEGKCVIDKVTRNQCQECRFKKCIYVGMATDLVLDQSKRLAKRKLIEENREK

SEQ ID NO: 21 - protein, 61 aa, Homo sapiens
GYHYGVWSCEQCKAFFKRSIQGHNDYMCPATNQCTIDKNRRKSCQACRLRKCYEVGMMKGG

SEQ ID NO: 22 - protein, 57 aa, Rattus sp.
GCHYGVLTCGSCKVFFKRAVEGQHNYLCAGRNDCIIDKIRRKNCPACRYRKCLQAGM

SEQ ID NO: 23 - protein, 20 aa, Homo sapiens
WAKRIPHFSELPLDDQVILL

SEQ ID NO: 24 - protein, 20 aa, T. cystophora
WAKRLPHFRDLSIADQVVLL

SEQ ID NO: 25 - protein, 20 aa, Homo sapiens
FAKKLPMFSELPCEDQIILL

SEQ ID NO: 26 - protein, 20 aa, Homo sapiens
FAKRLPGFTGLSIADQITLL

SEQ ID NO: 27 - protein, 20 aa, Homo sapiens
YAKSIPGFVNLDLNDQVTLL

SEQ ID NO: 28 - protein, 20 aa, Homo sapiens
FAKQLPGFLQLSREDQIALL

SEQ ID NO: 29 - protein, 20 aa, Homo sapiens
FAKMIPGFRDLTSEDQIVLL

SEQ ID NO: 30 - protein, 20 aa, Homo sapiens
WAKRVPGFVDLTLHDQVHLL

SEQ ID NO: 31 - protein, 20 aa, Homo sapiens
WAKAIPGFRNLHLDDQMTLL

SEQ ID NO: 32 - protein, 20 aa, Homo sapiens
WSKSLPGFRNLHIDDQITLI

SEQ ID NO: 33 - protein, 9 aa, Homo sapiens
LLLRLPALR

SEQ ID NO: 34 - protein, 9 aa, T. cystophora
VILRIPALR

SEQ ID NO: 35 - protein, 9 aa, Homo sapiens
LLMKVTDLR

SEQ ID NO: 36 - protein, 9 aa, Homo sapiens
MLMKITDLR

SEQ ID NO: 37 - protein, 9 aa, Homo sapiens
LLQKMTDLR

SEQ ID NO: 38 - protein, 9 aa, Homo sapiens
MLMKLVSLR

SEQ ID NO: 39 - protein, 9 aa, Homo sapiens
MIQKLADLR

SEQ ID NO: 40 - protein, 9 aa, Homo sapiens
LLLILSHIR

SEQ ID NO: 41 - protein, 9 aa, Homo sapiens
LTKLLDSMH

SEQ ID NO: 42 - protein, 9 aa, Homo sapiens
LTKLLDNLH

SEQ ID NO: 43 - protein, 6 aa, Homo sapiens
FLMEML

SEQ ID NO: 44 - protein, 6 aa, T. cystophora
FLLDML

SEQ ID NO: 45 - protein, 6 aa, Homo sapiens
LFLEVF

SEQ ID NO: 46 - protein, 6 aa, Homo sapiens
LIREML

SEQ ID NO: 47 - protein, 6 aa, Homo sapiens
LLQEIY

SEQ ID NO: 48 - protein, 6 aa, Homo sapiens
LLSEIW

SEQ ID NO: 49 - protein, 6 aa, Homo sapiens
LVLEVF

SEQ ID NO: 50 - protein, 6 aa, Homo sapiens
LLLEML

SEQ ID NO: 51 - protein, 6 aa, Homo sapiens
MLAEII

SEQ ID NO: 52 - protein, 6 aa, Homo sapiens
MVSEVI

(19) United States
(12) Patent Application Publication
(10) Pub. No.: US 2003/0003517 A1
(43) Pub. Date: Jan. 2, 2003

(54) Methods of detecting dissociated nuclear hormone receptor ligands

(76) Inventors: Elliott S. Klein, Danbury, CT (US); Weizhen Wang, Newport Coast, CA (US); Roshantha A. Chandraratna, Laguna Hills, CA (US)

Correspondence Address: CAMPBELL & FLORES LLP, 4370 LA JOLLA VILLAGE DRIVE, 7TH FLOOR, SAN DIEGO, CA, 92122, US

(21) Appl. No.: 09/814,604

(22) Filed: Mar. 22, 2001

(51) Int. Cl. G01N 33/567

(52) U.S. Cl. 435/7.21

(57) ABSTRACT

The present invention provides a method of identifying an effective agent that dissociates nuclear hormone receptor activities. The method includes the steps of contacting a nuclear hormone receptor with one or more agents under conditions suitable for forming a test complex containing nuclear hormone receptor dimer, coactivator and corepressor; assaying for coactivator association with the test complex; and assaying for corepressor association with the test complex, where coactivator association combined with corepressor association indicates that at least one of the agents is an effective agent that dissociates nuclear hormone receptor activities.

### BACKGROUND OF THE INVENTION

1. Field of the Invention

The present invention relates generally to the fields of biochemistry and molecular medicine and, in particular, to drugs that result in simultaneous corepressor and coactivator interaction with nuclear hormone receptor complexes.

1. Background Information

Nuclear hormone receptors are a large family of gene regulatory, DNA-binding proteins that bind hormonally and nutritionally derived lipophilic ligands. Over 300 nuclear hormone receptors have been identified to date, including, for example, the retinoid X receptor, retinoic acid receptor, progesterone receptor, estrogen receptor, androgen receptor and vitamin D receptor (Whitfield et al., J. Cell. Biochem. Suppl. 32/33:110-122 (1999); Laudet et al., Cell 97:161-163 (1999); and Sluder et al., Genome Res. 9:103-120 (1999)). Nuclear hormone receptors have been conserved throughout evolution and play a role in cell growth and proliferation, development and homeostasis. Not surprisingly, nuclear hormone receptors have been implicated in disease. Retinoic acid receptors can play a role in, for example, acute promyelocytic leukemia and acne; thyroid hormone receptor is involved in thyroid hormone resistance and hypercholesterolemia; vitamin D receptors play a role in type 2D-dependent rickets and osteoporosis; peroxisome proliferator activated receptor (PPAR) contributes to obesity and Type II diabetes; and the estrogen receptor plays a role in some forms of breast cancer (Lazar, J. Invest. Medicine 47:364-368 (1999)). Progress has been made in understanding the role of nuclear hormone receptors and their ligands in disease, and in identifying hormone receptor ligands with therapeutic activity.

In the case of the retinoid receptors, retinoid ligands have been developed as therapeutics for a variety of disorders. Current retinoid therapies include differentiation of acute promyelocytic leukemia (APL); treatment of nodulocystic acne, a severe form of inflammatory acne; treatment of psoriasis; prevention of secondary head and neck cancers; topical therapy of acne vulgaris; and reversal of UV-mediated photodamage (Thacher et al., Current Pharm. Design 6:25-58 (2000)). Unfortunately, the dosage of these retinoid ligands is limited by significant side effects, including irritation and inflammation of skin and mucous membranes, elevation of serum triglycerides, dysregulation of bone formation and resorption, headaches, hypothyroidism, and fetal malformation. Thus, there is a need for a new generation of retinoid and other hormone-based therapeutics which can have, for example, greater selectivity and fewer side effects.

Nuclear hormone receptors have long been known to be DNA-binding proteins that can activate or repress transcription of target genes. In most cases, transcriptional activity of the hormone receptor is controlled in a ligand-dependent manner. Current assays for identifying therapeutic ligands are based on the transcriptional activity of the nuclear hormone receptor of interest. However, compounds identified using these assays often are characterized by significant side effects.

Thus, there is a need for novel assays which can be used to identify therapeutic hormone receptor ligands but which do not rely on the transcriptional activity of the nuclear hormone receptor. The present invention satisfies this need and provides related advantages as well.

### SUMMARY OF THE INVENTION

The present invention provides a method of identifying an effective agent that dissociates nuclear hormone receptor activities. The method includes the steps of contacting a nuclear hormone receptor with one or more agents under conditions suitable for forming a test complex containing nuclear hormone receptor dimer, coactivator and corepressor; assaying for coactivator association with the test complex; and assaying for corepressor association with the test complex, where coactivator association combined with corepressor association indicates that at least one of the agents is an effective agent that dissociates nuclear hormone receptor activities. In a method of the invention, the test complex can be, for example, a ternary complex containing nuclear receptor dimer and bound cognate response element. In one embodiment, a nuclear hormone receptor is contacted with one or more agents in vitro. In another embodiment, the nuclear hormone receptor is contacted with one or more agents in the presence of a eukaryotic cell sample, which can contain, for example, viable cells, a whole cell lysate, or a fractionated cell lysate. In another embodiment, the eukaryotic cell sample contains an exogenous nucleic acid molecule encoding the nuclear hormone receptor. In a further embodiment, the coactivator or corepressor, or both, are provided in the eukaryotic cell sample.

A variety of nuclear hormone receptors are useful in the invention including, for example, a retinoid X receptor (RXR), hepatocyte nuclear factor 4 (HNF4), testicular receptor, tailless gene homolog (TLX), chicken ovalbumin upstream promoter transcription factor (COUP-TF), thyroid receptor (TR), retinoic acid receptor (RAR), peroxisome proliferator activated receptor (PPAR), reverse Erb (revErb), RAR-related orphan receptor (ROR), steroidogenic factor-1 (SF-1), liver receptor homolog-1 (LRH-1), liver X receptor (LXR), farnesoid X receptor (FXR), vitamin D receptor (VDR), ecdysone receptor (EcR), pregnane X receptor (PXR), constitutive androstane receptor (CAR), neuron-derived activated receptor (NOR1), nuclear receptor related 1 (NURR1), estrogen receptor (ER), estrogen-related receptor (ERR), glucocorticoid receptor (GR), androgen receptor (AR), progesterone receptor (PR) or mineralocorticoid receptor (MR). In one embodiment, the nuclear hormone receptor is a retinoic acid receptor, retinoid X receptor, thyroid receptor, estrogen receptor or peroxisome proliferator activated receptor. In a further embodiment, the nuclear hormone receptor is RARα, RARβ, RARγ, RXRα, RXRβ or RXRγ. In yet another embodiment, the nuclear hormone receptor is RARα, RARβ or RARγ.

A screening method of the invention can be practiced by assaying for any of a variety of coactivators. Such coactivators include, for example, SRC-1/NCoA-1; TIF2/GRIP-1/NCoA-2; ACTR/p/CIP/AIB1/NCoA-3; p300/CBP; p/CAF; or TATA box binding protein. In one embodiment, the coactivator is SRC-1/NCoA-1. The association of a corepressor also is assayed in a method of the invention; such a corepressor can be, for example, N-CoR or SMRT.

A variety of means can be used to assay for coactivator association and corepressor association in a method of the invention. Coactivator association can be assayed by specific binding to the test complex, for example, by immunoprecipitation of the test complex. In one embodiment, the immunoprecipitation is performed using antibody immunoreactive with the nuclear hormone receptor dimer. In another embodiment, coactivator association is assayed by immunodetection of the coactivator. Similarly, corepressor association can be assayed by specific binding to the test complex, for example, by immunoprecipitation of the test complex. Such immunoprecipitation can be performed, for example, using antibody immunoreactive with the nuclear hormone receptor dimer. In one embodiment, corepressor association is assayed by immunodetection of the corepressor.

### BRIEF DESCRIPTION OF THE DRAWINGS

FIG. 1 shows retention of N-CoR corepressor by the dominant negative RXR, RXRαΔC. Ternary complexes containing RARα/RXRα (lanes 1 and 2), RARα/RXRαΔC (lanes 3 and 4), RARα(AHT)/RXRα (lanes 5 and 6), and RARα(AHT)/RXRαΔC (lanes 7 and 8), were immunoprecipitated from transfected CV-1 whole cell extracts in the absence (lanes 1, 3, 5 and 7) or presence (lanes 2, 4, 6 and 8) of 1 μM TTNPB and increasing amounts of a DR-5 RARE (0-0.5 μg). Anti-SRC-1, N-CoR and RXRα antibodies were used to detect co-immunoprecipitated SRC-1, N-CoR or RXRα, respectively.

FIG. 2 shows effects of RAR mutations on N-CoR interaction with the ternary complex. (A) Ternary complexes containing RARα (lanes 1 and 3) and RARα R₂₇₂-A (lanes 2 and 4) were immunoprecipitated from transfected CV-1 whole cell extracts in the absence (lanes 1 and 2) or presence (lanes 3 and 4) of 1 μM TTNPB. (B) Ternary complexes containing wild type or mutant RARα were immunoprecipitated in the presence of vehicle (top panel), 1 μM TTNPB (middle panel), or 1 μM AGN196382 (bottom panel).

FIG. 3 shows that N-CoR retention effects the transactivation profile of RARα selective ligands. (A) CV-1 cells transfected with ER-RARα were treated with the indicated ligands. The percentage response represents luciferase activity expressed as a percentage of ATRA mediated transactivation, ±standard error of quadruplicate determinations normalized to β-galactosidase activity. (B) RARα containing ternary complexes were immunoprecipitated in the presence of the indicated ligands (1 μM). Anti-SRC-1, N-CoR, ACTR, p300 or RXRα antibodies were used to detect co-immunoprecipitated proteins as indicated.

FIG. 4 shows that the RARα mutant R₂₇₂-A exhibits decreased affinity for RAR ligand. CV-1 cells transfected with RARα-P-GR (solid symbols) or RARα(R₂₇₂-A)-P-GR (open symbols) were treated with either TTNPB (circle) or AGN196382 (square) at the indicated concentrations. The percentage luciferase activity expressed±standard error of quadruplicate determinations was normalized to β-galactosidase activity.

FIG. 5 shows the dose response of coregulator interactions with ternary complexes. (A) Ternary complexes containing RARα (lanes 1-5) and RARα R₂₇₂-A (lanes 6-10) were immunoprecipitated from transfected CV-1 whole cell extracts in the presence of the indicated concentration of TTNPB. (B) RARα containing ternary complexes were immunoprecipitated from transfected CV-1 whole cell extracts in the presence of increasing concentration of the indicated ligands. Lanes 2, 3, 4 and 5 represent 10⁻⁹, 10⁻⁸, 10⁻⁷, 10⁻⁶ Molar ATRA, respectively. The same dose response performed with AGN194794 is shown in lanes 6-9. The equivalent dose response performed with AGN196382 is shown in lanes 10-13.

FIG. 6 shows anti-AP1 activity of several RARα selective ligands.

FIG. 7 shows the amino acid sequence of human RAR isoforms. (A) The amino acid sequence of RARα (SEQ ID NO: 1). (B) The amino acid sequence of human RARβ (SEQ ID NO: 2). (C) The amino acid sequence of human RARγ (SEQ ID NO: 3).

FIG. 8 shows a schematic view of nuclear receptor functional domains. Modular diagrams in the top panel are drawn to scale and aligned at the conserved E1 domain. The DNA-binding region consists of two (Cys)₄-type zinc-finger motifs (C4 Zn fingers), followed by a C-terminal extension (CTE) of varying length. Dimerization and ligand-binding contacts determined by X-ray crystallography also are shown in the top panel. The center panel shows a selected portion of the DNA-binding domain for several receptors, with solid circles indicating DNA contacts as determined by X-ray crystallography for human RXRα, human TRβ, human ERα and rat GR. Jellyfish RXR is shown for comparison. The lower panel details three subregions of the ligand-binding domain in several nuclear hormone receptors, including the conserved E1 domain that supports dimerization and participates in transactivation; h9, which participates in dimerization; and the AF2 region, which contains ligand contacts and effects transactivation. Residues highly conserved among all nuclear hormone receptors are present in the E1 subregion and highlighted. SEQ ID NOS: are shown in parenthesis.

### DETAILED DESCRIPTION OF THE INVENTION

The present invention is directed to the surprising discovery that ligands or other compounds that induce the simultaneous association of a coactivator and corepressor with a nuclear hormone receptor can function to “dissociate” nuclear hormone receptor activities. Such ligands or compounds can have selective indirect effects on signaling pathways, such as anti-AP-1 or IFNγ/STAT activity, and on other nuclear hormone receptor-mediated pathways, while at the same time failing to directly activate transcription through the cognate response element to which the ligand-activated hormone receptor binds. Based on these findings, the invention provides novel screening methods for identifying nuclear hormone receptor ligands and regulators. Such screening methods can be useful for identifying improved ligands with reduced or minimal side effects, for example, for identifying retinoid ligands or compounds for treating cancer, acne, psoriasis and other dermatological disorders.

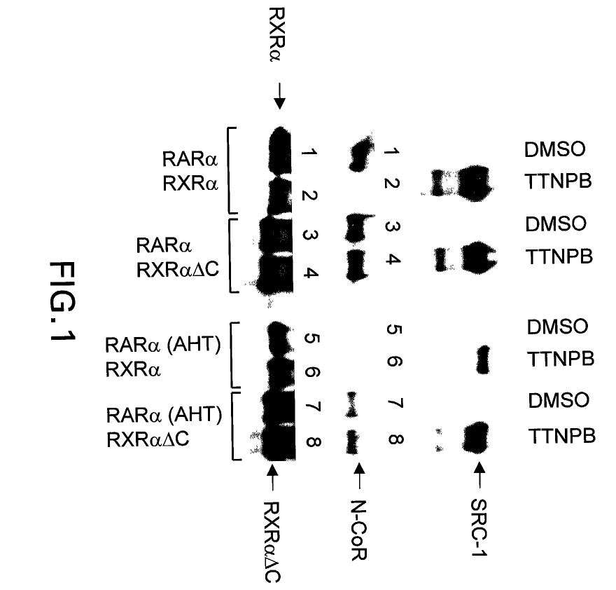

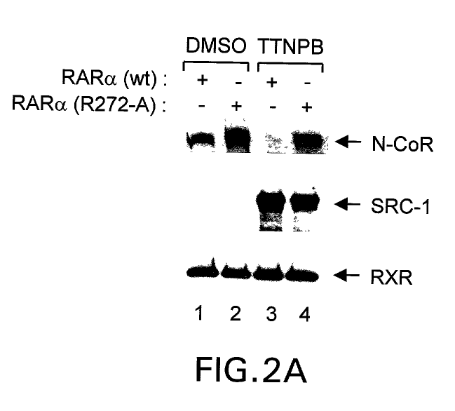

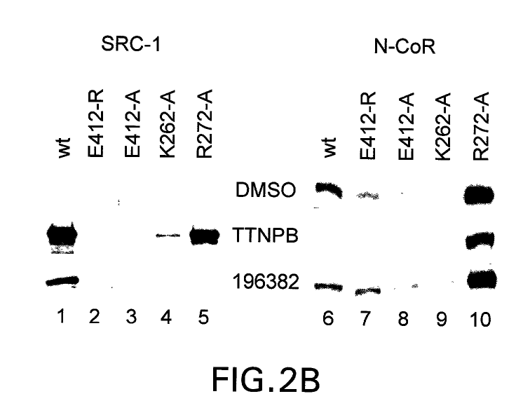

As disclosed herein in Example I, expression of wild type RARα with a dominant negative form of RXR truncated at amino acid 448 (RXRαΔC) resulted in a heterodimer that failed to release the corepressor N-CoR upon binding agonist TTNPB (see FIG. 1, lane 4 of middle panel). In contrast to the failure to release corepressor, the RARα/RXRαΔC heterodimer recruited coactivator SRC-1 effectively in response to agonist treatment (FIG. 1, top panel). Furthermore, mutation of RARα arginine 272 to alanine, proximal to the CoR box, resulted in a mutant in which TTNPB mediated SRC-1 recruitment was essentially normal while dissociation of N-CoR corepressor was severely impaired (see Example II and FIG. 2). Thus, these altered receptor forms display simultaneous interaction of coactivator and corepressor upon treatment with agonist.

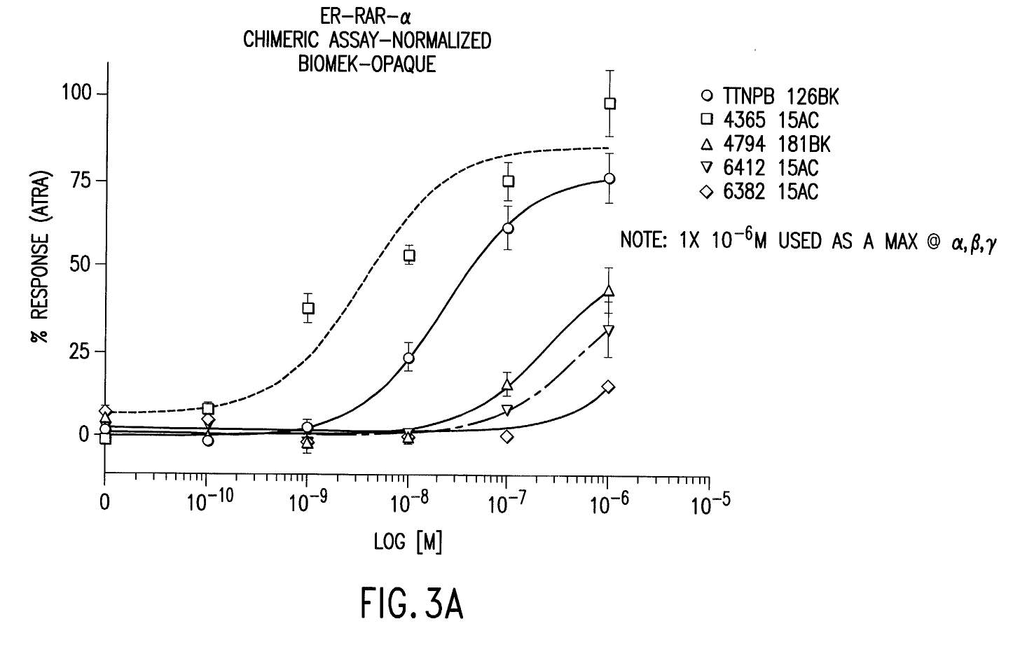

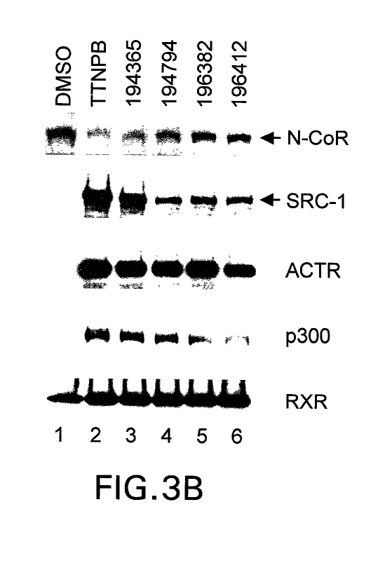

As further disclosed herein in Example IV, RARα selective ligands with similar binding affinities for RARα displayed disparate transactivation profiles. Specifically, while AGN194365 potently transactivated RARα and AGN194794 activated RARα with slightly less efficacy and potency, the compounds AGN196382 and 196412 had no activity at RARα except at the highest dose (1 μM) tested (see FIG. 3A). The disparate transactivation activity did not correlate with the amount of SRC-1 recruited to the ternary complex, as shown in FIG. 3B. However, dissociation of N-CoR was significantly impaired in response to either AGN196382 or AGN196412, the ligands with the weakest transactivation capabilities. This impaired N-CoR dissociation was not observed with the stronger transactivators, TTNPB, AGN194365 and AGN194794. These results indicate a direct correlation between failure to transactivate and failure to release N-CoR.

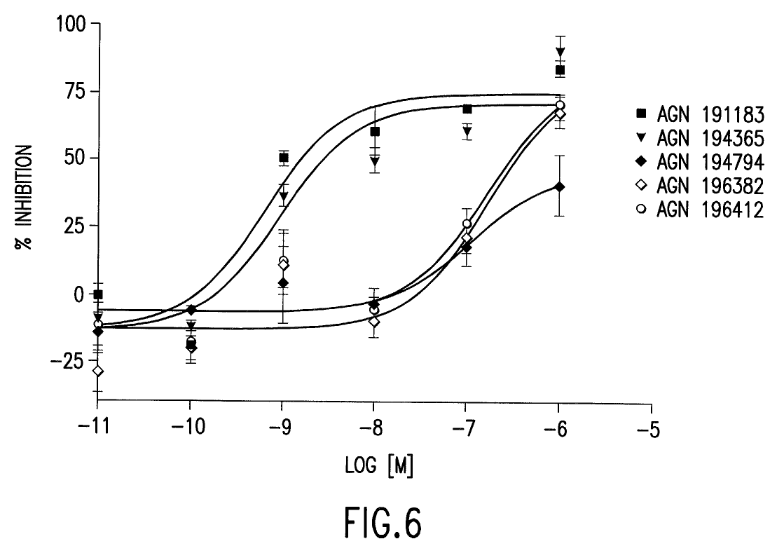

As further disclosed herein in Example VI, AGN196382 was tested for the ability to antagonize phorbol ester stimulated transcription from a collagenase-promoter construct containing an AP-1 response element. As shown in FIG. 6A, AGN196382 exhibited efficacious anti-AP1 activity at 10 μM, a dose at which transcriptional activation of RARα was not seen. These results demonstrate that hormone ligands can have selective indirect effects on signaling pathways, such as anti-AP-1 or IFNγ/STAT activity, while lacking the ability to directly activate transcription through a bound nuclear hormone receptor response element. In sum, these results indicate that dissociated ligands with selective activity can be identified based on their ability to induce coactivator recruitment and simultaneous corepressor retention.

Based on these discoveries, the present invention provides a method of identifying an effective agent that dissociates nuclear hormone receptor activities. The method includes the steps of contacting a nuclear hormone receptor with one or more agents under conditions suitable for forming a test complex containing nuclear hormone receptor dimer, coactivator and corepressor; assaying for coactivator association with the test complex; and assaying for corepressor association with the test complex, where coactivator association combined with corepressor association indicates that at least one of the agents is an effective agent that dissociates nuclear hormone receptor activities. In one embodiment, the test complex is a ternary complex containing nuclear receptor dimer and bound cognate response element. In another embodiment, the nuclear hormone receptor is contacted with one or more agents in vitro. In a further embodiment, the nuclear hormone receptor is contacted with one or more agents in the presence of a eukaryotic cell sample, which can contain, for example, viable cells, a whole cell lysate, or a fractionated cell lysate. In another embodiment, the eukaryotic cell sample contains an exogenous nucleic acid molecule encoding the nuclear hormone receptor. In yet a further embodiment, the coactivator or corepressor, or both, are provided in the eukaryotic cell sample.

A variety of nuclear hormone receptors are useful in the invention including, for example, a retinoid X receptor (RXR), hepatocyte nuclear factor 4 (HNF4), testicular receptor, tailless gene homolog (TLX), chicken ovalbumin upstream promoter transcription factor (COUP-TF), thyroid receptor (TR), retinoic acid receptor (RAR), peroxisome proliferator activated receptor (PPAR), reverse Erb (revErb), RAR-related orphan receptor (ROR), steroidogenic factor-1 (SF-1), liver receptor homolog-1 (LRH-1), liver X receptor (LXR), farnesoid X receptor (FXR), vitamin D receptor (VDR), ecdysone receptor (EcR), pregnane X receptor (PXR), constitutive androstane receptor (CAR), neuron-derived activated receptor (NOR1), nuclear receptor related 1 (NURR1), estrogen receptor (ER), estrogen-related receptor (ERR), glucocorticoid receptor (GR), androgen receptor (AR), progesterone receptor (PR) or mineralocorticoid receptor (MR). In one embodiment, the nuclear hormone receptor is a retinoic acid receptor, retinoid X receptor, thyroid receptor, estrogen receptor or peroxisome proliferator activated receptor. In a further embodiment, the nuclear hormone receptor is RARα, RARβ, RARγ, RXRα, RXRβ or RXRγ. In yet another embodiment, the nuclear hormone receptor is RARα, RARβ or RARγ.

A screening method of the invention can be practiced by assaying for any of a variety of coactivators. Such a coactivator can be, for example, SRC-1/NCoA-1; TIF-2/GRIP-1/NCoA-2; ACTR/p/CIP/AIB1/NCoA-3; p300/CBP; p/CAF; or TATA box binding protein. In one embodiment, the coactivator is SRC-1/NCoA-1. The association of a corepressor also is assayed in a method of the invention; such a corepressor can be, for example, N-CoR or SMRT.

A variety of means can be used to assay for coactivator association and corepressor association in a method of the invention. Coactivator association can be assayed by specific binding to the test complex, for example, by immunoprecipitation of the test complex. In one embodiment, the immunoprecipitation is performed using antibody immunoreactive with the nuclear hormone receptor dimer. In another embodiment, coactivator association is assayed by immunodetection of the coactivator. Similarly, corepressor association can be assayed by specific binding to the test complex, for example, by immunoprecipitation of the test complex. Such immunoprecipitation can be performed, for example, using antibody immunoreactive with the nuclear hormone receptor dimer. In one embodiment, corepressor association is assayed by immunodetection of the corepressor.

The methods of the invention relate to identifying an effective agent that dissociates nuclear hormone receptor activities. As used herein in reference to an effective agent and a given nuclear hormone receptor, the term “dissociates” means that the effective agent has selective activity on an indirect signalling pathway activated by the nuclear hormone receptor, while lacking or having significantly reduced direct gene transcription activity at genes regulated through cognate response elements. Such an indirect signalling pathway can be an AP-1 mediated pathway or STAT mediated pathway or a pathway mediated by another nuclear hormone receptor. For example, an effective agent identified by screening according to a method of the invention with a retinoic acid receptor can have significant anti-AP-1 activity while having relatively little or no direct transcriptional activity mediated by cognate RAR response elements.

A model of hormone activation of nuclear receptors, as exemplified for the retinoic acid receptor (RAR) and the thyroid hormone receptor (T3R), involves interaction of unliganded receptor with corepressor, resulting in observed trans-repression activity of these receptors. Upon hormone binding, interaction with corepressor is decreased in favor of interaction with coactivator. Corepressors and coactivators exhibit, or are associated with proteins exhibiting, histone deacetylase or histone acetyl-transferase activity, respectively, indicating that ligand regulated nuclear receptors control access of the transcriptional machinery to chromatin. Recruitment of corepressors upon binding of antagonists or inverse agonists has been demonstrated for ER, PPAR, and RAR. These results indicate that the nature of the ligand modulates nuclear hormone receptor interaction with coactivator or corepressor molecules.

Corepressor release can be a prerequisite for the increased transcriptional activity of several hormone nuclear receptors upon agonist binding. Examples of nuclear receptor variants which fail to release corepressor exhibit aberrant function and can be responsible for various human disorders (Barroso et al., Nature 402:880-883 (1999)). Dominant negative variants of several nuclear receptors have been described (Gurnell et al., J. Biol. Chem. 275:5754-5759 (2000); and Berger et al., Mol. Cell. Endocrin. 162:57-67 (2000)); several of these dominant negative variants have been demonstrated to share a similar phenotype of corepressor retention in spite of ligand addition. Similarly, the PML-RARα fusion protein homodimer, resulting from the 15;17 chromosomal translocation characteristic of all-trans retinoic acid (ATRA) responsive acute promyelocytic leukemia (APL), exhibits corepressor retention in the presence of ATRA at normal physiological concentrations and corepressor release only at higher, pharmacological concentrations. The resulting lack of PML-RARα transactivation at physiological ATRA concentrations results in proliferation of myeloid precursors which fail to terminally differentiate. In addition, patients exhibiting resistance to thyroid hormone have been identified which express thyroid hormone receptor β (TRβ) harboring point mutations. Several of these, which occur near the previously described CoR box located within helix 1 of the ligand binding domain, result in impaired dissociation of corepressor from TRβ in the presence of thyroid hormone and hyporesponsiveness to elevated thyroid hormone levels.

The p160 coactivator family member SRC-1 contains a centrally located receptor interaction domain (RID). This domain, which is conserved among the other members of this family of coactivator proteins, contains three amphipathic, α-helical “NR boxes” or “LXDs” containing the core amino acid sequence “LXXLL.” Analysis of a homodimer of a PPARγ ligand binding domain co-crystalized with an 88 amino acid polypeptide of SRC-1 containing NR boxes 2 and 3 indicates that each NR box makes contact with one coactivator binding site in a PPAR monomer, providing a stoichiometry of one coactivator to one homodimer. Similarly, mutation analysis of the corepressor, N-CoR, has demonstrated that two LXXLL-like sequences, LXXIIXXXL (SEQ ID NO: 4), are used for nuclear receptor interaction. Furthermore, mutations in the helices that make up the coactivator binding site in TRβ result in loss of corepressor interaction, indicating that coactivators and corepressors share a overlapping interaction domain (Nagy et al., Genes Dev. 13:3209-3216 (1999)). Similar to that proposed for coactivator, a model has been proposed whereby the each of the two LXXIIXXXL (SEQ ID NO: 4) motifs of N-CoR make contact with one monomer of a RAR/RXR or T3R/RXR heterodimer (Perissi et al., Genes Devel. 13:3198-3208 (1999)).

A variety of coactivators are known in the art and useful in the methods of the invention. Such coactivators are proteins that generally promote ligand-dependent transcriptional activation of nuclear hormone receptors and, in some cases, other transcription factors, and typically are associated with chromatin remodeling due to associated histone acetyltransferase activity. One class of coactivator complex includes the SRC-1/NCoA-1/p160 family (steroid receptor coactivator-1/nuclear receptor coactivator-1) family members (Onate et al., Science 270:1354-1357 (1995)). SRC-1 family members and other coactivators can interact with nuclear hormone receptors containing the C-terminal activation domain 2 (AF-2). The AF-2 interaction is highly ligand dependent and can occur through direct binding to the minimal activation domain of AF-2, denoted AF-2AD, which has been mapped to the C-terminal α-helix 12 (H12) in the ligand binding domain (Glass and Rosenfeld, Genes Dev. 14:121-141 (2000)). Additional coactivators include TIF-2/GRIP-1/NCoA-2 (transcriptional intermediary factor-2/glucocorticoid receptor interacting protein-1/nuclear receptor coactivator-2; Chakravarti et al., Nature 383:99-103 (1996)) and ACTR/p/CIP/AIB1 (Anzick et al., Science 277:965-968 (1997)), which also interact in a ligand dependent manner with the AF-2 activation domain.

CBP/p300 (CREB-binding protein/p300 (Chen et al., Cell 98:675-686 (1999)) is a coactivator that serves as an essential coactivator not only for nuclear receptors but also for other transcription regulatory factors. CBP/p300, like the SRC-1 family members, has histone acetyltransferase activity (Spencer et al., Nature 389:194-198 (1997)). Additional coactivator complexes include TRAP/DRIP (TRAP220/DRIP205/PBP), which has multiple components and interacts in a ligand-dependent manner with helix 12 in the ligand binding domain (Rachez et al., Nature 398:824-828 (1999); and Zhu et al., J. Biol. Chem. 272:25500-25506 (1997)). Addition coactivators useful in the invention include p/CAF (p300/CBP-associated factor; Blanco et al., Genes Devel. 12:1638-1651 (1998); and Korzus et al., Science 279:703-707 (1998)).

As used herein, the term “coactivator” means a protein that forms a complex with nuclear hormone receptor in a ligand-dependent manner and which exhibits increased association with nuclear hormone receptor upon agonist treatment. Coactivators useful in the invention include but are not limited to the following: SRC-1/NCoA-1; TIF-2/GRIP-1/NCoA-2; ACTR/p/CIP/AIB1/NCoA-3; p300/CBP; p/CAF; and TATA box binding protein (TBP). A coactivator can have histone acetyltransferase activity or be associated with a protein having histone acetyltransferase activity, and recruitment of a coactivator to a nuclear hormone receptor can be required for transactivation through cognate response element. It is understood that coactivators may associate with some but not all nuclear hormone receptors and, furthermore, that coactivators can associate with other transcription factors in addition to nuclear hormone receptors. One skilled in the art further understands that a coactivator can bind directly or indirectly to the nuclear hormone hreceptor or its heterodimeric partner.

The term coactivator encompasses a fragment, fusion protein or variant of a naturally occurring coactivator, provided that the fragment, fusion protein or variant retains at least one receptor binding region and associates with the nuclear hormone receptor in a ligand (agonist) dependent manner. One skilled in the art understands that a coactivator, or variant or fragment thereof, typically retains at least one “NR” box, which contains the core amino acid sequence “LXXLL,” and can contain two or more such boxes.

The term “corepressor,” as used herein, means a protein that forms a complex with nuclear hormone receptor in a ligand-dependent manner and which exhibits decreased association with nuclear hormone receptor upon agonist treatment. Corepressors useful in the invention include without limitation N-CoR and SMRT. A corepressor can have histone deacetyltransferase activity or be associated with a protein having histone deacetyltransferase activity, and release of corepressor from a nuclear hormone receptor complex can be required for transactivation by the receptor. One skilled in the art understands that a corepressor may associate selectively with some but not all nuclear hormone receptors and, furthermore, that some corepressors can associate with other transcription factors in addition to nuclear hormone receptors. It further is understood that a corepressor can bind directly to a nuclear hormone receptor or its heterodimeric partner, or can bind indirectly to a nuclear hormone receptor by binding another associated component of the receptor-containing complex.

The term corepressor, as used herein, encompasses a fragment, fusion protein or variant of a naturally occurring corepressor, provided that the fragment, fusion protein or variant retains at least one receptor binding region and dissociates from the nuclear hormone receptor in a ligand (agonist) dependent manner. A corepressor, or fragment, fusion protein or variant thereof useful in the invention, typically retains at least one “LXXIIXXXL” sequence, and can retain two or more of such sequences.

The methods of the invention rely on a nuclear hormone receptor, which can be endogenous or exogenous, transiently or stably introduced into cultured cells, or provided as a purified or partially purified protein, for example, protein recombinantly expressed and purified from host cells such as bacterial, insect or mammalian cells, as described further below. A variety of nuclear hormone receptors can be useful in the methods of the invention including, for example, a retinoid X receptor (RXR), hepatocyte nuclear factor 4 (HNF4), testicular receptor, tailless gene homolog (TLX), chicken ovalbumin upstream promoter transcription factor (COUP-TF), thyroid receptor (TR), retinoic acid receptor (RAR), peroxisome proliferator activated receptor (PPAR), reverse Erb (revErb), RAR-related orphan receptor (ROR), steroidogenic factor-1 (SF-1), liver receptor homolog-1 (LRH-1), liver X receptor (LXR), farnesoid X receptor (FXR), vitamin D receptor (VDR), ecdysone receptor (EcR), pregnane X receptor (PXR), constitutive androstane receptor (CAR), neuron-derived activated receptor (NOR1), nuclear receptor related 1 (NURR1), estrogen receptor (ER), estrogen-related receptor (ERR), glucocorticoid receptor (GR), androgen receptor (AR), progesterone receptor (PR) or mineralocorticoid receptor (MR).

In one embodiment, a nuclear hormone receptor used in a method of the invention binds as a homodimer to its cognate response element. Such a nuclear hormone receptor can be, for example, a glucocorticoid, estrogen, androgen, progestin, or mineralocorticoid receptor. In another embodiment, a nuclear hormone receptor used in a method of the invention binds as a heterodimer to its cognate response element. Such a nuclear hormone receptor can be, for example, a retinoic acid receptor, thyroid receptor, vitamin D receptor, farnesoid X receptor, oxysterol receptor, peroxisome proliferator receptor or ecdysone receptor, each of which bind as a heterodimer with the retinoid X receptor.

In a further embodiment of the invention, the nuclear hormone receptor is a retinoid X receptor, retinoic acid receptor, progesterone receptor, estrogen receptor, androgen receptor or vitamin D receptor. In yet a further embodiment of the invention, the nuclear hormone receptor is a retinoic acid receptor such as RARα, RARβ or RARγ.

In its native form, a nuclear hormone receptor is a polypeptide which (1) contains a DNA-binding domain; (2) contains a ligand-binding domain; and (3) is localized in its naturally occurring environment, at least in part, to the nucleus of eukaryotic cells. A native nuclear hormone receptor generally has a DNA-binding domain containing two (Cys)₄ zinc finger motifs, and most often is a ligand-dependent transcription factor, for example, a ligand-dependent transcriptional activator. It is recognized that a nuclear hormone receptor may reside in the cytoplasm in the absence of ligand, translocating at least in part to the nucleus or other cellular compartment upon ligand-binding as in the case of the glucocorticoid and mineralocorticoid receptors. Thus, nuclear localization of a nuclear hormone receptor can be ligand-dependent. Nuclear hormone receptors useful in the invention include full length steroid hormone receptors; thyroid/retinoid/vitamin D and peroxisome proliferator activated receptors; and orphan receptors, and fragments of these receptors.

Native nuclear hormone receptors generally share a similar domain structure. An N-terminal extension of varying length often harbors a transactivation function (AF1), for example, in steroid receptors such as the estrogen and progesterone receptors. A well-conserved central DNA binding region typically contains two zinc-finger DNA binding motifs of the (Cys)₄ type. A variable C-terminal extension (CTE) flanks the zinc fingers and participates in DNA binding by some receptors, for example, thyroid receptor. A large C-terminal ligand binding domain (LBD) also is seen in nuclear hormone receptors, generally having ligand contacts in three distinct clusters and separate from receptor dimerization contacts that also occur in the ligand binding domain. The conserved E1 subregion, as well as a less well-conserved heptad nine (h9) region and a second transactivation domain (AF2) also lie within the ligand binding domain.

Native nuclear hormone receptors typically dimerize, either as a homodimer or as a heterodimer, for example, with RXR or USP, followed by high-affinity binding to specific hexanucleotide half-elements arranged in a particular motif. Many nuclear hormone receptors bind DNA in one of the following patterns: (1) as heterodimers with RXR (or USP) on directly (tandemly) repeated half elements separated by a spacer of 1-5 bp; (2) as heterodimers on inverted (palindromic) response elements separated by 1 bp; (3) as homodimers on direct repeats separated by 1 bp; (4) as homodimers on inverted repeats separated by 3 bp; or (5) as monomers on a single half-site, which may contain a 3 bp 5′ extension. The hexanucleotide half-element generally is a variation of AGGTCA, although several steroid receptors such as the glucocorticoid receptor, mineralocorticoid receptor, progesterone receptor and androgen receptor bind an AGAACA half-site. Exemplary heterodimers include RXR/RAR; RXR/VDR; RXR/LXR; RXR/PXR; RXR/CAR and PPAR/RXR, each of which bind to direct repeats, and RXR/FXR and USP/EcR, each of which bind to inverted half-repeats. Exemplary homodimers include glucocorticoid, estrogen, androgen and mineralocorticoid receptor homodimers, each of which bind to palindromic repeats separated by 3 bp. While both receptors of a homodimer likely are liganded for activity, liganding of the primary receptor residing on the 3′ half-element generally is sufficient for activity of a heterodimer (Whitfield, supra, 1999).

A variety of nuclear hormone receptors are known in the art, and these receptors as well as fragments, fusion proteins and variants of naturally occurring receptors are useful in the invention as described further below. See, for example, Mangelsdorf et al., Cell 83:835-9 (1995); Enmark and Gustafsson, Mol. Endocrinol. 10:1293-1307 (1996); Kumar and Thompson, Steroids 64: 310-319 (1999); and Whitfield et al., supra, 1999. Nucleic acid sequences encoding human and other mammalian, vertebrate and non-vertebrate nuclear hormone receptors readily can be obtained from a variety of sources, for example, from databases such as GenBank. For example, a nucleic acid sequence encoding human RARα is available as GenBank accession AF088890; a nucleic acid sequence encoding human RARβ is available as GenBank accession NM_{—}000965; and a nucleic acid sequence encoding human RARγ is available, for example, as GenBank accession M38258. Similarly, a nucleic acid sequence encoding human RXRα is available as GenBank accession NM_{—}002957; a nucleic acid sequence encoding human RXRβ is available as GenBank accession AF065396; and a nucleic acid sequence encoding human RXRγ is available, for example, as GenBank accession NM_{—}006917. Nucleic acid sequences encoding a variety of additional nuclear hormone receptors also are known in the art and readily available to the skilled person; sources for exemplary nucleic acid sequences useful in the invention are provided in Table 1. These sources and sequences are hereby incorporated by reference herein.

TABLE 1

| | GenBank |
|---|---|
| Nuclear Hormone Receptor | accession |
| human retinoid X receptor α (hRXRα) | NM_002957 |
| human retinoid X receptor β (hRXRβ) | AF065396 |
| mouse retinoid X receptor β (mRXRβ) | D21830 |
| human retinoid X receptor γ (hRXRγ) | NM_006917 |
| mouse retinoid X receptor γ (mRXRγ) | S62948 |
| jellyfish retinoid X receptor | AF091121 |
| human ovalbumin upstream promoter | NM_005654 |
| transcription factor | |
| human thyroid receptor α (hTRα) | X55070 |
| mouse thyroid receptor β (mTRβ) | U15544 |
| Xenopus thyroid receptor β | U04675 |
| Fugu fish thyroid receptor α | AJ012380 |
| human retinoic acid receptor α (hRARα) | AF088890 |
| human retinoic acid receptor β (hRARβ) | NM_000965 |
| human retinoic acid receptor γ (hRARγ) | M38258 |
| human peroxisome proliferator activated | AB005523 |
| receptor γ (hPPARγ) | |
| mouse RAR-related orphan receptor γ | AF019657 |
| human steroidogenic factor-1 (hSF-1) | S65878 |
| human vitamin D receptor (hVDR) | AB002162 |
| Drosophila ecdysone receptor | M74078 |
| mouse constitutive androstane receptor | AF009326 |
| α | |
| human nuclear receptor related 1 | AB019433 |
| (hNURR1) | |
| mouse nuclear receptor related 1 | AAC53153 |
| (mNURR1) | |
| human estrogen receptor α (hERα) | AF123494 |
| Atlantic salmon estrogen receptor α | AF047895 |
| tilapia fish estrogen receptor α | X93558 |
| human glucocorticoid receptor (hGR) | U78508 |
| human androgen receptor (hAR) | M27423 |
| human progesterone receptor (hPR) | X69071 |
| chicken progesterone receptor (cPR) | M32726 |
| human mineralocorticoid receptor (hMR) | AF068617 |

As used herein, the term “nuclear hormone receptor” means a polypeptide containing the ligand binding domain of a nuclear hormone receptor. Such a nuclear hormone receptor retains the ability to bind a known ligand of one of the nuclear hormone receptors referenced in Table 1, or contains a ligand binding domain exhibiting substantial amino acid homology to the ligand binding domain of one of the receptors referenced in Table 1, or both. Where a nuclear hormone receptor is one of the receptors shown in Table 1, the receptor retains the ability to bind a known ligand with a binding constant (K_{d}) of at least 300 nM, and can bind, for example, with a K_{d} of at least 200 nM, 100 nM, 75 nM, 50 nM, or higher.

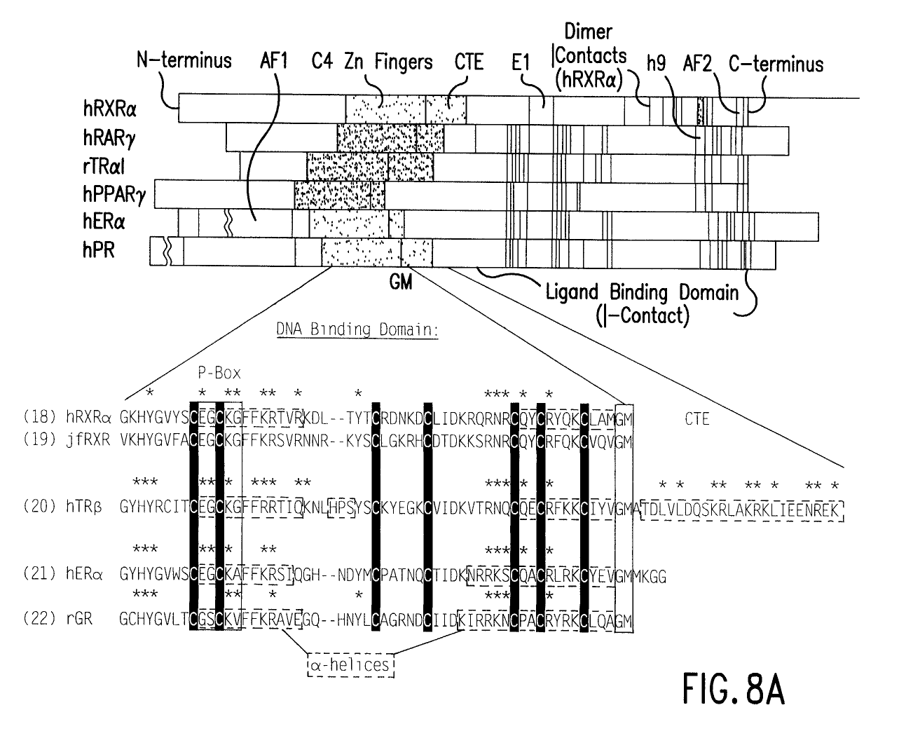

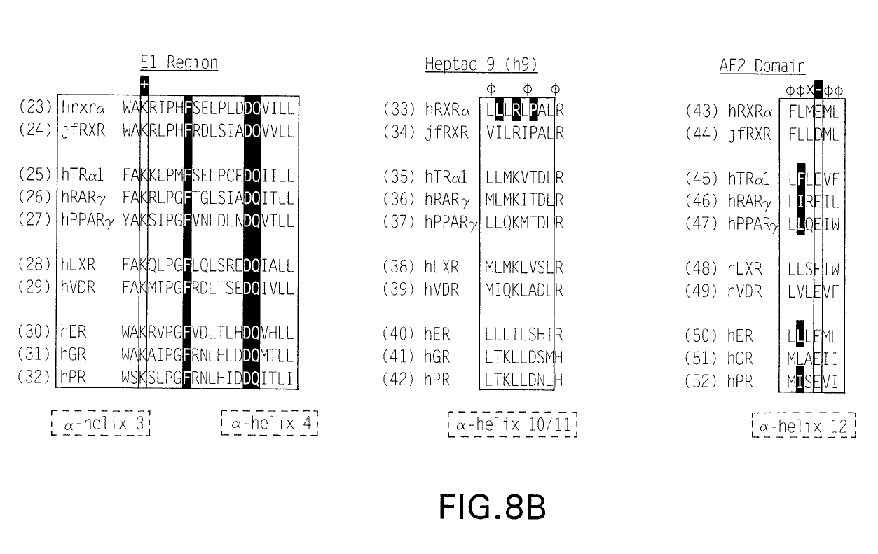

Within its ligand binding domain, a nuclear hormone receptor can exhibit primary, secondary or tertiary structural homology to at least one of the ligand-binding domains of the nuclear hormone receptors referenced in Table 1 and generally has a tertiary structure which is a sandwich of 11 to 13 α-helices and several small β-strands organized around a lipophilic binding cavity (Williams and Sigler, Nature 393:392-396 (1998)). A nuclear hormone receptor ligand-binding domain generally contains three subregions: a conserved E1 domain; the heptad 9 (h9) subregion; and an AF2 subregion (Whitfield et al., supra, 1999). A nuclear hormone receptor can be recognized, for example, as a protein containing the conserved lysine, phenylalanine and aspartic acid-glutamine residues in the E1 subregion as shown in FIG. 8.

The term nuclear hormone receptor encompasses polypeptides having an amino acid sequence that is identical to the wild type hormone receptor sequence, and polypeptides having a similar, non-identical sequence that is considered by those skilled in the art to be a functionally equivalent amino acid sequence. An RAR receptor, for example, can have an amino acid sequence identical to one of the RAR isoforms (SEQ ID NOS: 1, 2 and 3), or a similar, non-identical sequence that is functionally equivalent.

It is understood that limited modifications can be made without destroying the biological function of a nuclear hormone receptor useful in the invention. Minor modifications of human RARU (SEQ ID NO: 1) that do not destroy ligand binding activity fall within the definition of RAR. Similarly, minor modifications of human retinoid X receptor α that do not destroy ligand binding fall within the definition of retinoid X receptor α, and minor modifications of human estrogen receptor that do not destroy estrogen binding activity fall within the definition of an estrogen receptor.

It is understood that minor modifications of primary amino acid sequence can result in polypeptides which have substantially equivalent or enhanced function as compared to wild type sequences, for example, compared to the wild type human RARα, β and γ sequences set forth herein. These modifications can be deliberate, as through site-directed mutagenesis, or can be accidental such as through mutation in hosts harboring an encoding nucleic acid. All such modified polypeptides are included in the definition of the particular nuclear hormone receptor as long as ligand binding activity or substantial sequence similarity are retained. It further is understood that various moieties can be attached to a nuclear hormone receptor, for example, a retinoid X receptor, retinoic acid receptor, progesterone receptor, estrogen receptor, androgen receptor or vitamin D receptor; such moieties include other polypeptides, carbohydrates, lipids, or chemical moieties. These fusion polypeptides or polypeptide conjugates also can be used in the screening methods of the invention as described further below.

Retinoids exert their biological effects through one or both of two families of nuclear hormone receptors, retinoic acid receptors (RARs) and retinoid X receptors (RXRs). Retinoic acid receptors and retinoid X receptors are ligand-dependent transcription factors which regulate gene transcription by both upregulating gene expression through binding RA-responsive elements and down-regulating gene expression by antagonizing the enhancer action of other transcription factors such as AP1. Distinct RXRα, RXRβ and RXRγ isotypes and RARα, RARβ and RARγ isotypes are encoded by separate genes. Both RXR and RAR isotypes can be further expressed as several isoforms. RAR isoforms differ in the N-terminal A region; these isoforms are generated by alternative splicing or differential usage of two promoters. Like other nuclear hormone receptors, in their native form RAR and RXR receptors are localized at least in part to the nucleus and contain DNA-binding, and ligand binding domains (see above). While RXR homodimers are responsive to RXR-activating compounds, the RXR subunit can be a silent partner in some heterodimers; for example, synthetic RXR agonists do not activate the RAR/RXR heterodimer.

All trans-retinoic acid is the physiological hormone for the RAR receptors and does not bind the RXR receptors. 9-cis-retinoic acid, a RXR receptor ligand, also binds to the RAR receptors. Various RXR and RAR specific synthetic ligands have been synthesized. For example, LG100268, AGN192599, SR11217, and SR11237 are RXR specific synthetic retinoids binding to all three RXRs but not to any of the RAR isotypes. TTNPB is an RAR-specific synthetic retinoid that binds RARs but not RXRs. RAR-selective ligands include AGN190299, a RARβ/γ selective ligand, and Am 580 and Am 80, which are RARα-selective in in vitro binding assays. Thus, RXR and RAR ligands can be selective or non-selective and can be naturally occurring or synthetic.

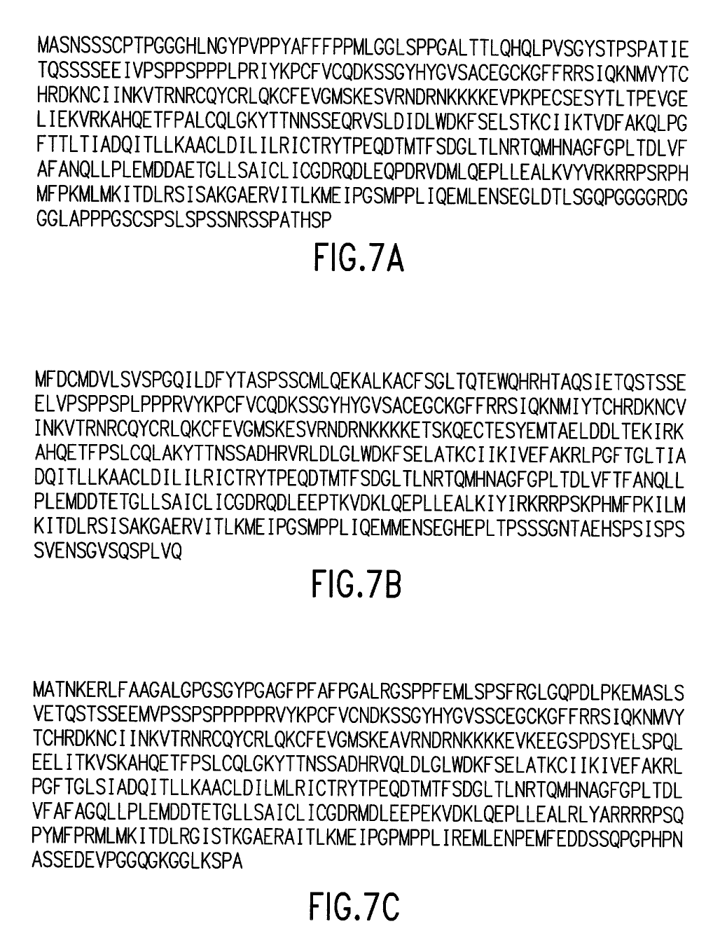

A method of the invention can be advantageously practiced with a retinoic acid receptor. The term “retinoic acid receptor” is synonymous with “RAR” and means a polypeptide which contains a ligand binding domain that binds a known retinoic acid receptor ligand, for example, TTNPB. A retinoic acid receptor can have the ligand binding domain of one of the naturally occurring human RAR isoforms such as human RARα (SEQ ID NO: 1), human RARβ (SEQ ID NO: 2) or human RARγ (SEQ ID NO: 3) shown in FIG. 7 and is intended to include related polypeptides having a ligand binding domain with substantial amino acid sequence similarity to the ligand binding domain of one of the RAR isoforms provided herein as SEQ ID NO: 1, 2 or 3. Such related polypeptides generally exhibit greater sequence similarity to the ligand binding domains of RARα, RARβ or RARγ than to the ligand binding domains of other nuclear hormone receptors containing (Cys)₄-type zinc finger motifs and include alternatively spliced forms of human RARα, RARβ or RARγ; species homologs including mouse, rat, primate and other mammalian homologs, vertebrate homologs and non-vertebrate homologs; and isotype variants of the amino acid sequences shown herein, provided that the polypeptide retains the ability to bind a known retinoic acid receptor ligand such as TTNPB. In one embodiment, a retinoic acid receptor contains a ligand binding domain having at least 75% amino acid identity with the ligand binding domain of SEQ ID NO: 1. In other embodiments, a retinoic acid receptor contains a ligand binding domain having at least 80%, 85%, 90% or 95% amino acid identity with the ligand binding domain of SEQ ID NO: 1. A retinoic acid receptor typically binds a known retinoic acid receptor ligand with a binding constant (K_{d}) of at least 300 nM, and can bind, for example, with a K_{d} of at least 200 nM, 100 nM, 75 nM, 50 nM, or higher.

The term nuclear hormone receptor, as used herein, also encompasses variants of wild type nuclear hormone receptors, for example, truncated nuclear hormone receptors, receptors containing one or more point mutations, or fusion proteins containing one or more heterologous hormone receptor or non-hormone receptor sequences in addition to the primary nuclear hormone receptor sequence. In one embodiment, a nuclear hormone receptor used in a screening method of the invention is a truncated nuclear hormone receptor containing the ligand-binding domain. In another embodiment, the nuclear hormone receptor is a polypeptide in which the nuclear hormone receptor sequences consist essentially of the DNA binding and ligand binding domains. In a further embodiment, the nuclear hormone receptor is a polypeptide in which the nuclear hormone receptor sequences consist essentially of the ligand binding domains. It is understood that a nuclear hormone receptor ligand binding domain, alone, can be fused, if desired, to one or more heterologous sequences. Similarly, the nuclear hormone receptor DNA binding and ligand binding domains can be fused to one or more heterologous sequences.

As used herein, the term “truncated nuclear hormone receptor” is synonymous with “truncated receptor” and means a deletion derivation of a wild type nuclear hormone receptor that lacks a portion of the wild type nuclear hormone receptor polypeptide sequence. A truncated nuclear hormone receptor can contain an N-terminal, internal or C-terminal deletion, or a combination thereof, and generally lacks 20 or more contiguous amino acids as compared to the wild type hormone receptor. A truncated nuclear hormone receptor can have a deletion of, for example, 50 or more, 100 or more, 150 or more, 200 or more, 250 or more, or 300 or more amino acids as compared to wild type hormone receptor.

A screening method of the invention can be practiced, for example, using a truncated nuclear hormone receptor consisting essentially of the ligand-binding domain. Such a receptor contains a functional ligand-binding domain and may contain various nuclear hormone receptor flanking residues adjacent to this domain but does not any other complete or functional nuclear hormone receptor domains such as the transactivation domain “A/B” or DNA-binding domain “C.” A ligand-binding domain of a nuclear hormone receptor generally is characterized, in part, as a sandwich of 11 to 13 α-helices and several small β-strands organized around a lipophilic binding cavity (Williams and Sigler, supra, 1998). A ligand-binding domain of a nuclear hormone receptor also contains three subregions: the conserved E1 domain, which includes conserved phenylalanine and aspartic acid-glutamine residues and can participate in dimerization and transactivation; the heptad 9 (h9) subregion, which can mediate, in part, dimerization; and the AF2 subregion, which can contain ligand contacts and effect transactivation (Whitfield et al., supra, 1999). Thus, it is recognized that a nuclear hormone receptor consisting essentially of a ligand-binding domain contains, in part, the AF2 subregion. It is understood that a truncated nuclear hormone receptor consisting essentially of the ligand-binding domain can be fused to one or more heterologous sequences, for example, a heterologous hormone receptor DNA binding domain or other DNA binding domain, for use in a screening method of the invention.

A variety of truncated nuclear hormone receptors consisting essentially of the ligand-binding domain can be used in a method of the invention. Such nuclear hormone receptor ligand-binding domains are well known in the art; for example, residues 229 to 387 of GenBank accession XM_{—}008647 encodes a human RARα ligand-binding domain; residues 719 to 829 of GenBank accession XM_{—}006190 encodes a human progesterone receptor ligand-binding domain; and 231 to 393 of GenBank accession NM_{—}000376 encodes a human vitamin D receptor ligand-binding domain. It is understood that these and additional nuclear hormone receptor ligand-binding domains are known in the art, or can be determined by comparison to known ligand-binding domains by routine methods.

A nuclear hormone receptor for use in a screening method of the invention can be a fusion protein containing a heterologous peptide or polypeptide sequence from a different nuclear hormone receptor, or from a protein that is not a nuclear hormone receptor. Such a fusion protein can contain, for example, a heterologous epitope tag or a heterologous DNA binding domain, in addition to the nuclear hormone receptor or truncated portion thereof.

As used herein, the term “heterologous” means a domain, tag or sequence derived from a different gene than the gene encoding the fused nuclear hormone receptor. Thus, for example, in a RARα-V5-AHT fusion protein, the “V5” tag is a heterologous epitope tag, which is not found in the gene encoding RXRα.

In one embodiment, a nuclear hormone receptor is expressed as a fusion protein containing a heterologous epitope tag, which can provide a convenient means for isolating a test complex and assaying for associated coactivator or corepressor. A variety of heterologous epitope tags are well known in the art and readily available including V5, FLAG, hemagluttinin (HA), c-myc, 6-HIS and AU1 tags. The FLAG tag DYKDDDDK (SEQ ID NO: 5), for example, can be used as an epitope tag (see Chubet and Brizzard, BioTechniques 20:136-141 (1996)). Well known heterologous epitope tags further include the HA tag YPYDVPDYA (SEQ ID NO: 6); the c-Myc epitope EQKLISEEDL (SEQ ID NO: 7); the AU1 tag DTYRYI (SEQ ID NO: 8); and the 6-HIS tag HHHHHH (SEQ ID NO: 9). One skilled in the art understands that these and other heterologous epitope tags can be fused to a nuclear hormone receptor, or to a coactivator or corepressor for use in a method of the invention.

A nuclear hormone receptor also can be expressed as a fusion with a heterologous DNA binding domain, which can be derived from a heterologous nuclear hormone receptor or another DNA binding protein. Exemplary chimeric fusion proteins useful in the methods of the invention include fusion proteins containing a estrogen receptor DNA binding domain fused to a RARα ligand binding domain or a GAL4 DNA binding domain fused to a RARα ligand binding domain.

In addition to practicing the methods of the invention with a nuclear hormone receptor, these methods can be practiced with a member of the steroid hormone receptor superfamily. The steroid hormone receptor superfamily includes the nuclear hormone receptors and additionally encompasses non-nuclear receptors which may or may not have a DNA-binding domain. Members of the steroid hormone receptor superfamily generally contain a ligand-binding domain with primary, secondary or tertiary structural homology to the ligand-binding domain of nuclear hormone receptors and generally have a tertiary structure which is a sandwich of 11 to 13 α-helices and several small β-strands organized around a lipophilic binding cavity. Three subregions are generally included in such a ligand binding domain: a conserved E1 domain; the heptad 9 (h9) subregion; and an AF2 subregion (Whitfield et al., supra, 1999). A member of the steroid hormone receptor superfamily generally is characterized, in part, by containing conserved lysine, phenylalanine and aspartic acid-glutamine residues in the E1 subregion as shown in FIG. 8. In addition, a polypeptide that binds a nuclear hormone receptor ligand or ligand of a member of the steroid hormone receptor superfamily is, itself, a member of the steroid hormone receptor superfamily.

In a method of the invention, a nuclear hormone receptor is contacted with one or more agents. As used herein, the term “agent” means any organic molecule, for example, a small molecule chemical; a peptide, peptidomimetic or peptoid; a protein, which can be an antibody or antigen-binding fragment thereof or a non-antibody protein; a nucleic acid molecule, for example, an oligonucleotide; an oligosaccharide; a lipoprotein; a glycolipid; or a lipid. Both naturally occurring and synthetic agents can be screened in a method of the invention. Naturally occurring agents are a product of nature in that the groups making up the molecule and the bonds linking the groups are produced by normal metabolic processes.

Agents to be screened generally are small lipophilic molecules that can diffuse across the plasma membrane and into cells freely. These molecules can be, for example, naturally occurring or synthetic retinoids (analogs of retinoic acid), eicosanoids, steroids, terpene-derived molecules and amino acid derivatives.

If desired, a population of agents can be assayed for activity en masse or in pools. For example, to identify an effective agent that dissociates RARα activities, cells transfected with a RARα-encoding nucleic acid molecule can be contacted with a population of agents and assayed for coactivator and corepressor associated with RARα; the active population can be subdivided and the assay repeated in order to isolate the effective agent from the population. Such screening protocols, in which compounds are assayed in pools of 10, 50, 100, 200, 500, 1000 or 10,000, for example, are well within the ability of those skilled in high throughput and ultra high throughput screening technology.

The methods of the invention rely on assaying for coactivator association combined with corepressor association to identify an effective agent that modulates a biological activity of the nuclear hormone receptor of interest. One skilled in the art understands that an “effective agent” that modulates a biological activity of a nuclear hormone receptor can reduce, enhance or change a biological activity of the nuclear hormone receptor either directly or indirectly and can be, for example, a precursor of an active compound, or a ligand of the nuclear hormone receptor.

To identify an effective agent according to a method of the invention, a nuclear hormone receptor is contacted with one or more agents. As used herein, the term “contacting” encompasses addition of the one or more agents to a lysate or combination of purified or partially purified proteins; addition of the one or more agents to a culture dish, flask or microtiter plate; and oral administration, injection, microinjection, infusion, or implantation of a slow release medium containing the one or more agents to be tested into an animal. Concentrations of agents to be tested generally are in the 10⁻¹² to 10⁻⁵ molar range and can be, for example, in the 10⁻⁹ to 10⁻⁶ molar range.

A method of the invention can be practiced with purified proteins or with a cell sample supplying one or any combination of the nuclear hormone receptor, coactivator and corepressor. A cell sample useful in the invention can be, for example, one of a variety of eukaryotic cell samples, including viable cells, which can be, for example, transiently or stably transfected cells; a whole cell lysate; or a fractionated cell lysate. A variety of eukaryotic cells are useful in the methods of the invention, including primary and immortalized cells, and a variety of cell types such as fibroblasts and adipocytes. A eukaryotic cell sample also can be prepared from a tumor cell, for example, a melanoma, colon tumor, breast tumor, prostate tumor, glioblastoma, renal carcinoma, neuroblastoma, lung cancer, bladder carcinoma, plasmacytoma or lymphoma cell. Where the nuclear hormone receptor is an RAR, RXR or combination thereof, convenient cell types are, for example, the human embryonic kidney cell line HEK293, the human cell line HeLa and the green monkey cell line CV-1. It is understood that the cell sample also can supply, if desired, an endogenous or exogenous heterodimeric partner of the nuclear hormone receptor to be assayed.

A cell sample such as a eukaryotic cell sample useful in the invention can be prepared from transiently or stably transfected cells, or from an animal expressing an exogenous nuclear hormone receptor, coactivator or corepressor. Methods for stably or transiently introducing a vector or nucleic acid molecule into a eukaryotic cell are well known in the art and include calcium phosphate transfection, electroporation, microinjection, DEAE-dextran and lipofection methods (see, for example, Ausubel, supra, 2000). A viral vector also can be useful to express an exogenous nuclear hormone receptor, coactivator or corepressor in a eukaryotic cell. Such a viral vector can be, for example, a retroviral vector, adenoviral vector, Herpes simplex virus vector, vaccinia virus vector, cytomegalovirus vector, Moloney murine leukemia virus vector, lentivirus vector, adeno-associated virus vector, or the like.

Expression of a nucleic acid molecule encoding a nuclear hormone receptor in vivo can be carried out using one of numerous methods well known in the art including adenoviral transformation, retroviral transformation, ballistic gun delivery, lentiviral transformation, cytomegaloviral transformation, and microinjection.

Where a nuclear hormone receptor, coactivator or corepressor is provided in purified or partially purified form, the receptor or variant can be produced routinely using recombinant methods or by chemical or proteolytic cleavage of the isolated polypeptide. Methods for chemical and proteolytic cleavage and for purification of the resultant hormone receptors are well known in the art as described, for example, in Deutscher, Methods in Enzymology, Vol. 182, “Guide to Protein Purification,” San Diego: Academic Press, Inc. (1990).

Nucleic acid sequences encoding nuclear hormone receptors, coactivators and corepressors can be used, for example, to prepare transiently or stably transfected cells or to prepare recombinant purified protein. As described above, nucleic acid sequences encoding a variety of nuclear hormone receptors are well known in the art and available for preparation of recombinant protein, as summarized in Table 1. Nucleic acid sequences encoding coactivators and corepressors also are well known in the art and available for recombinant expression. For example, the amino acid and corresponding nucleic acid sequence encoding human SRC-1/NCoA-1 are available as GenBank accession U40396 (Onate et al., supra, 1995); the amino acid and corresponding nucleic acid sequence encoding human TIF2/GRIP-1/NCoA-2 are available as GenBank accession X97674 (Voegel et al., EMBO J. 15:3667-3675 (1996); the amino acid and corresponding nucleic acid sequence encoding human ACTR/p/CIP/AIB1/ NCoA-3 are available as AF036892 (Chen et al., Cell 90:569-580 (1997); and the amino acid and corresponding nucleic acid sequence encoding human TRAP220/DRIP205/PBP are available as AF283812 (Rachez et al., Nature 398:824-828 (1999). Similarly, the nucleic acid and corresponding amino acid sequence encoding human N-CoR are available as NM_{—}006311 (Horlein et al., supra, 1995). It is understood that these are exemplary nucleic acid sequences illustrating that a variety of coactivator and corepressors have been cloned and their sequences available to the skilled person. Additional coactivator and corepressor sequences are well known in the art or can be isolated by routine methods; such sequences include, but are not limited to, species homologs and proteins related in sequence to the coactivator and corepressors described herein.

A variety of means can be used to assay for coactivator association and corepressor association in a method of the invention, and it is understood that one can assay for coactivator association prior to, simultaneous with, or after assaying for corepressor association. Coactivator association can be assayed by specific binding to the test complex, for example, by immunoprecipitation of the test complex. In one embodiment, the immunoprecipitation is performed using antibody immunoreactive with the nuclear hormone receptor dimer, and coactivator association then is assayed by immunodetection of the coactivator. Similarly, corepressor association can be assayed by specific binding to the test complex, for example, by immunoprecipitation of the test complex. Such immunoprecipitation can be performed, for example, using antibody immunoreactive with the nuclear hormone receptor dimer, and corepressor association can be assayed subsequently by immunodetection of the corepressor.

Where the test complex is not in isolated form, assaying for coactivator or corepressor association can include isolating the test complex, which contains nuclear hormone receptor dimer. Isolation can be performed by specific binding to the test complex, for example, by specific binding to the nuclear hormone receptor component of the test complex. In one embodiment, the test complex conveniently is isolated by immunoprecipitation.

Further, where the components of the test complex are not provided in purified form, the test complex conveniently can be isolated by expressing the nuclear hormone receptor as a fusion protein with a heterologous epitope tag. Convenient heterologous epitope tags include V5, FLAG, hemagluttinin (HA), c-myc, 6-HIS and AU1 epitope tags. For example, fusion proteins containing the FLAG tag DYKDDDDK (SEQ ID NO: 5) can be produced by routine molecular methods; anti-FLAG monoclonal antibodies are commercially available from, for example, Eastman Kodak (Rochester, N.Y.) and Berkeley Antibody Company (BabCO; Richmond, Calif.), and polyclonal serum is available from Santa Cruz Biotechnology (Santa Cruz, Calif.). The HA tag YPYDVPDYA (SEQ ID NO: 6) can be engineered into a recombinant nuclear hormone receptor or other component, and anti-HA antibody or antiserum obtained from BabCO, Roche Diagnostics (Indianopolis, Ind.) or Santa Cruz Biotechnology. Similarly, one can engineer the c-Myc epitope EQKLISEEDL (SEQ ID NO: 7), which is recognized by antibody or antisera commercially available from BabCO, Invitrogen (San Diego, Calif.), Roche Diagnostics, SIGMA (St. Louis, Mo.) or Santa Cruz Biotechnology. Addition epitope tags useful in the invention include the AU1 tag DTYRYI (SEQ ID NO: 8), which is recognized by a monoclonal antibody available from BabCO, and the 6-HIS tag HHHHHH (SEQ ID NO: 9), which is recognized by antibodies and antisera available, for example, from BabCO, Invitrogen, SIGMA or Santa Cruz Biotechnology. If desired, a fusion protein containing a 6-HIS epitope can be purified using metal chelate chromatography (see Ausubel et al., supra, 10.15, Supplement 41). One skilled in the art understands that these and other epitope tags can be conveniently used to isolate a test complex in a method of the invention.

Immunoaffinity purification can be performed to isolate a test complex using, for example, antibody or antisera immunoreactive with an epitope of the nuclear hormone receptor; antibody or antisera immunoreactive with an epitope of a heterodimeric partner or other component of the test complex, or can be performed using antibody or antisera immunoreactive with a heterologous epitope tag fused, for example, to the nuclear hormone receptor or its dimeric partner. In one embodiment, immunoprecipitation is performed using antibody immunoreactive with the nuclear hormone receptor dimer. In another embodiment of the invention, a heterologous epitope tag is fused to the nuclear hormone receptor, and an antibody or antisera that is immunoreactive with the epitope tag is used to isolate the test complex.

Affinity purification, including immunoaffinity, DNA affinity, and other types of affinity purification, can be used to isolate a test complex. In one embodiment, a nuclear hormone receptor or other component of the test complex is expressed as a fusion protein in a form suitable for affinity purification, for example, as a fusion with glutathione S transferase (GST). To produce such a fusion protein, a nuclear hormone receptor can be cloned into a pGEX vector (Amersham Pharmacia; Piscataway, N.J.) for expression as a C-terminal fusion protein with glutathione S transferase; expressed in bacteria; and subsequently purified using affinity to glutathione agarose (Ausubel, supra, 2000; Chapter 16 and Supplement 28). After contacting purified GST-receptor or a lysate containing the GST-receptor fusion protein with one or more agents under conditions suitable for forming a test complex containing nuclear hormone receptor dimer, coactivator and corepressor, the test complex can be isolated using affinity purification, for example, with glutathione-agarose (Ausubel, supra, 2000, Chapter 20 and Supplement 33).

Immunoprecipitation can be conveniently used to isolate a test complex in order to assay for coactivator or corepressor association with nuclear hormone receptor. As used herein, the term “immunoprecipitation” means any process by which an antigen or antigen-containing complex is isolated by binding to a specific antibody attached to a sedimentable matrix. Immunoprecipitation is performed by addition of a specific antibody to a sample that includes the test complex; the specific antibody can be polyclonal antisera, or one or more monoclonal antibodies, and is attached to a sedimentable matrix, which can be, for example, protein A or protein G-agarose beads, or Sepharose. It is recognized that the polyclonal or monoclonal antibody can specifically bind, for example, a native nuclear hormone receptor epitope; an epitope of a heterodimeric receptor partner or other protein associated with the nuclear hormone receptor; or can specifically bind a heterologous epitope tag fused, for example, to the receptor or its heterodimeric partner. Low-speed centrifugation typically is performed to separate the solid-phase matrix and bound proteins, and washing is performed to remove unbound proteins. Exemplary conditions for immunoprecipitating a nuclear hormone receptor using an epitope tagged-hormone receptor fusion protein are disclosed herein in Example I. In addition, a variety of immunoprecipitation protocols are well known in the art, as described, for example, in Harlow and Lane, Antibodies: A Laboratory Manual (Cold Spring Harbor Laboratory Press, 1988); and Ausubel, supra, 2000 (see especially and Chapter 10 (Supplement 48) and Chapter 20 (Supplement 46)).

The term “immunodetection,” as used herein in reference to a coactivator or corepressor, means a process utilizing a detectable antibody or antigen-binding fragment thereof, which specifically binds the coactivator, corepressor, or a heterologous epitope. It is recognized that the antibody or antigen binding fragment thereof can be detected directly or indirectly, as described further herein below.

A variety of means can be used to assay for association of a coactivator or corepressor with a nuclear hormone receptor. In one format, the test complex is immunoprecipitated with an antibody having specificity for a nuclear hormone receptor monomer component thereof from the mixture provided in step (a). The immunoprecipitate can be subjected, for example, to polyacrylamide electrophoresis, and the separated proteins transferred to a suitable membrane for immobilization, such as nitrocellulose, and probed with an antibody having specificity for the coactivator or corepressor. A signal above background indicates that there is association with the nuclear hormone receptor. If desired, the results can be compared to an immunoprecipitate from a lysate or other sample similarly treated but without the one or more agents to be tested; an increase in the amount of coactivator or corepressor detected by the antibody indicates association of the coactivator or corepressor with the nuclear hormone receptor.

An association also can be assayed by incubating together a test complex, for example, a ternary complex, and the one or more agents to be assayed, then permitting the test complex and any associated coactivator or corepressor proteins to specifically bind a solid support. The immobilized complex can be washed, and presented with a first antibody having binding specificity for a coactivator such as SRC-1 and a second antibody having binding specificity for a corepressor, for example, N-CoR. Detection of the antibody having binding specificity for the coactivator and detection of the antibody having binding specificity for the corepressor serve to assay for association of coactivator or corepressor, respectively. The results can optionally be compared to signals obtained from a test complex not treated with the one or more agents. An increase in the amount of coactivator association as compared to a test complex not treated with the one or more agents combined with an increase in the amount of corepressor association as compared to the control complex not treated with the one or more agents indicates that at least one of the agents is an effective agent that dissociates nuclear hormone receptor activities.

The methods of the invention can be automated, if desired. A cell-free lysate, for example, optionally including a cognate response element, can be incubated with a panel of test compounds in separate wells of a microtiter dish (such as 96 well plates), then transferred via a robotic pipetting device to a fresh microtiter dish containing wells having an interior surface coated with an antibody specific for a nuclear hormone receptor. Washing can be performed by automated pipetting and shaking or mixing of the microtiter dishes. Similar to an ELISA (enzyme-linked immunosorption assay) format, a labeled antibody having specificity for a coactivator can be added to each well using the automated pipetting device, the antibody permitted to bind, and then the well washed free of unbound label. In the same or a separate well, a labeled antibody having specificity for a corepressor is added to each well. After binding, the well is washed free of unbound label. The antibodies can be linked to an enzyme and detected with a spectrophotometer after reacting with a chromogenic substrate. Where both coactivator and corepressor are detected in the same well, it is understood that two different detection signals must be used.

A variety of detection methods are useful in the methods of the invention. A reagent such as an antibody can be labeled, for example, with a radioisotope, luminescent compound (including a chemiluminescent compound such as an acridinium ester or a derivative), a fluorescent compound, biotin, iminobiotin, avidin, an electron dense component, a magnetic component, an enzyme, a hormone component, or a metal-containing component. Methods of detecting such labels include, without limitation, spectrophotometry, luminometry, nuclear magnetic resonance, autoradiography, scintillation counting and the like.

An antibody useful in immunodetection, immunoprecipitation, or other immunoaffinity purification can be, for example, can be polyclonal or monoclonal, or a pool of monoclonal antibodies, and, furthermore, can be a polypeptide fragment of an antibody that retains a specific binding activity for a nuclear hormone receptor, coactivator, corepressor, or heterologous epitope tag of at least about 1×10⁵ M⁻¹. One skilled in the art would know that antibody fragments such as Fab, F(ab′)₂ and Fv fragments can retain specific binding activity and, thus, can be useful in the invention. It further is understood that immunodetection, immunoprecipitation or other immunoaffinity purification can be performed with a non-naturally occurring antibody or fragment containing, at a minimum, one V_{H} and one V_{L} domain, for example, a chimeric antibody, humanized antibody or single chain Fv fragment (scFv) that specifically binds a nuclear hormone receptor, coactivator or corepressor. Such a non-naturally occurring antibody can be constructed using solid phase peptide synthesis, produced recombinantly or obtained, for example, by screening combinatorial libraries consisting of variable heavy chains and variable light chains as described by Borrebaeck (Ed.), Antibody Engineering (Second edition) New York: Oxford University Press (1995)).

A variety of antibodies and antisera useful in the invention can be obtained commercially. For example, rabbit anti-RXRα is available from Santa Cruz Biotech; mouse anti-SRC-1 is available from Affinity BioReagents; and goat anti-N-CoR is available from Santa Cruz Biotech. A variety of antibodies that bind heterologous epitopes also are commercially available. For example, antibodies that recognize FLAG, hemagluttinin (HA), c-myc, AU1 and 6-HIS tag are commercially available, for example, from BabCO, Invitrogen, Roche Diagnostics, SIGMA or Santa Cruz Biotechnology.

An antibody or antiserum useful in the invention also can be prepared by routine methods, for example, using a nuclear hormone receptor, coactivator, or corepressor, or a synthetic peptide encoding a portion of one of these proteins as an immunogen. One skilled in the art would know that purified nuclear hormone receptor, coactivator or corepressor, which can be produced recombinantly, or fragments of these proteins, including peptide portions such as synthetic peptides, can be used as an immunogen. Non-immunogenic fragments or synthetic peptides of a nuclear hormone receptor or other protein can be made immunogenic by coupling the hapten to a carrier molecule such as bovine serum albumin (BSA) or keyhole limpet hemocyanin (KLH). In addition, various other carrier molecules and methods for coupling a hapten to a carrier molecule are well known in the art are described, for example, by Harlow and Lane, supra, 1988.

In the methods of the invention, a nuclear hormone receptor is contacted with one or more agents under conditions under conditions suitable for forming a test complex that contains nuclear hormone receptor dimer, coactivator and corepressor. Thus, the nuclear hormone receptor is combined with at least one coactivator and at least one corepressor. The coactivator can be endogenously or recombinantly expressed in a cell in which the nuclear hormone receptor is expressed or can be endogenously or recombinantly expressed in a eukaryotic cell sample which is combined with another source of nuclear hormone receptor. Similarly, at least one corepressor can be endogenously or recombinantly expressed in a cell in which the nuclear hormone receptor is expressed or can be endogenously or recombinantly expressed in a eukaryotic cell sample which is combined with nuclear hormone receptor.

In one embodiment, at least one coactivator or corepressor is provided in substantially purified form. In another embodiment, the nuclear hormone receptor, coactivator and corepressor each are provided in substantially purified form. For example, nuclear hormone receptor can be expressed as a fusion protein with an epitope tag or as a fusion protein with GST. Following purification from bacteria using routine methods, bacterially expressed GST alone or GST-nuclear hormone receptor fusion protein can be bound to glutathione-Sepharose 4B beads (Amersham Pharmacia Biotechnology). One or more coactivators and one or more corepressors also can be expressed in purified or partially purified form, and, if desired, can be expressed with a detectable tag, such as an epitope tag, or radiolabeled subsequent to purification. For example, a coactivator or corepressor can be translated in vitro and labeled with ³⁵S-methionine. The ³⁵S-labeled or otherwise detectable proteins can be combined with GST-beads or GST-nuclear hormone receptor beads in the presence or absence of the one or more agents to be tested. After incubation, the beads are washed to remove free protein and the bound beads extracted with loading buffer, separated by SDS-PAGE and assayed by autoradiography.

In one embodiment, the present invention provides a method of identifying an effective agent that dissociates nuclear hormone receptor activities by contacting a nuclear hormone receptor with one or more agents under conditions suitable for forming a test complex containing nuclear hormone receptor dimer, coactivator and corepressor; assaying for coactivator association with the test complex as compared to a control complex; and assaying for corepressor association with the test complex as compared to a control complex, where coactivator association combined with corepressor association indicates that at least one of the agents is an effective agent that dissociates nuclear hormone receptor activities. The control complexes for comparison of coactivator association and corepressor association can be the same control complexes or can be different. In another embodiment, the assay for coactivator association with the test complex is compared to a first control complex treated with vehicle or a reference agent that does not induce coactivator recruitment, and the assay for corepressor association with the test complex is compared to a second control complex treated with an agonist of the nuclear hormone receptor or a reference agent known to cause corepressor release. An increase in coactivator association of the test complex as compared to the first control complex, and an increase in corepressor association of the test complex as compared to the second control complex indicates that at least one of the agents is an effective agent that dissociates nuclear hormone receptor activities.

The term control complex, as used herein, refers to a complex that is not contacted with the one or more agents to be assayed. A control complex generally is contacted with vehicle or with reference agent having a known effect on coactivator or corepressor association to the nuclear hormone receptor.

To prepare a control complex, one skilled in the art would use a corresponding cell, lysate, or purified or partially purified protein, or combination thereof, and would culture the cells, if used, under the same conditions as the cells from which the test complex is prepared. Thus, the control complex preferably has the same amount and type of nuclear hormone receptor and the same amount and type of heterodimeric partner. Where cells or tissue are used, the cells and tissue are preferably of the identical cell or tissue type used to prepare the test complex. Even more preferably, cells or tissue of the identical cell or tissue type are grown under the same conditions as the cells or tissue from which the test complex is prepared. As disclosed herein, for example, a cell lysate prepared from CV-1 cells transiently transfected with RXRα and RARα-V5 was contacted with the test RAR selective ligands; as a control, the same CV-1 cell lysate was treated with control vehicle (DMSO) and, separately, a compound known to recruit coactivator and release corepressor (the PAR agonist, TTNPB). One skilled in the art understands that transiently transfected CV-1 cells or another immortalized cell line expressing a similar level of RARα and RXRα can be used to prepare a control complex. One skilled in the art further understands that a control complex can be assayed for coactivator or corepressor association before, after, or simultaneously with an assay performed to determine association of coactivator and corepressor with the test complex, or can be determined, if desired, by referencing a historical value.

The control complex generally is contacted with vehicle or a reference agent having a known effect. To compare coactivator association, one skilled in the art can compare, for example association in the test complex to a control complex not treated at all, or treated with vehicle such as DMSO. Given that very little SRC-1 associates with nuclear hormone receptor treated with DMSO, an increased signal is an indication of the association of coactivator with the test complex. To compare corepressor association, a convenient control is an agonist of the nuclear hormone receptor known to induce corepressor release. For example, TTNPB treatment of the RAR receptor results in release of N-CoR. Where the nuclear hormone receptor is RAR, an increase in corepressor association with the test complex as compared to TTNPB treated control complex is indicative of corepressor association.

The rate and extent of coactivator and corepressor binding to nuclear hormone receptors can be greatly increased upon the prior formation of a ternary complex containing the receptor dimer and a nucleic acid containing the cognate response element as compared to the rate and extent of binding of coactivators and corepressors in the absence of the response element. In one embodiment, a method of the invention relies on a ternary complex that includes nuclear receptor dimer and bound cognate response element. Exemplary preparation of such a ternary complex is provided herein in Example I, which discloses formation of a ternary complex formed from CV-1 cell extract expressing recombinant RARα and RXRα with up to 0.5 μg double stranded synthetic DR-5 RARE cognate response element added prior to ligand addition and incubated on ice for 30 minutes prior to assaying for association of coactivator and corepressor (FIG. 1). See, also, Klein et al., J. Biol. Chem. 275: 19401-19408 (2000).

The term “ternary complex,” as used herein, means specifically associated nuclear hormone receptor dimer and bound cognate response element. It is understood that the ternary complex can contain a nuclear hormone receptor homodimer or heterodimer and further can contain one or more additional specifically associated proteins such as coactivators and corepressors.

As used herein, the term “cognate response element” means a nucleic acid molecule that specifically binds both monomers of a given nuclear hormone receptor dimer. A cognate response element useful in the invention typically contains two short hexanucleotide half elements separated by one or more variable nucleotides. A cognate response element can be a naturally occurring or synthetic sequence. A cognate nuclear hormone receptor response element can contain two half elements specific for the same nuclear hormone receptor, such as the RAR DR-5 response element (“RARE;” 5′-GGTTCAN₅AGTTCA-3′ (SEQ ID NO: 10). Where the cognate response element is bound by a heterodimer, the response element contains two half elements specific for different nuclear hormone receptors.

A variety of cognate response elements are known in the art, and additional response elements can be defined, for example, using routine in vitro gel retardation assays or transfection assays and known agonists of the nuclear hormone receptor. Cognate response elements binding a retinoic acid receptor (RAR) are denoted RAREs and have been divided into three classes: Class I RAREs are arranged as direct hexanucleotide repeats separated by 5 random nucleotides (“DR-5” response elements). Class II RAREs are response elements having direct repeats separated by two nucleotides (denoted “DR-2” response elements). Class III RAREs are RAR cognate response elements that are neither DR-5 or DR-2 response elements, often having a more complex structure. This class includes response elements having inverted repeats, repeats separated by no nucleotides or up to 14 nucleotides, and response elements containing three half element repeats. Exemplary RAR cognate response elements include the DR-5 element 5′-GGTTCAN₅AGTTCA-3′ (SEQ ID NO: 10), the DR-2 element 5′-AGGTCAN₂AGGTCA-3′ (SEQ ID NO: 11), and the class III element 5′-AGGTGAN₃AGGTTAN₁₄GGGTCA-3′ (SEQ ID NO: 12). A variety of additional RAR cognate response elements are known in the art, as described, for example, in Nagpal and Chandraratna, Current Pharm. Design 2:295-316 (1996).

RXR cognate response elements typically have two direct repeats separated by a single nucleotide although some RXR cognate response elements contain up to five direct repeats. Exemplary RXR cognate response elements are provided herein as 5′-AGGGCAN₁AGGTCA-3′ (SEQ ID NO: 13) and 5′-CGGCAAN₁AGGTCA-3′ (SEQ ID NO: 14). These and additional RXR cognate response elements are known in the art (see Nagpal and Chandraratna, supra, 1996)). Cognate response elements for other nuclear hormone receptors also are known in the art. For example, the RXR/VDR heterodimer binds the VDRE 5′-GGGTCAN₃GGTTCA-3′ (SEQ ID NO: 15) and glucocorticoid homodimer bind the palindromic GRE 5′-AGAACAN₃TGTTCT-3′ (SEQ ID NO: 16).

It is understood that, where the nuclear hormone receptor is a chimera that contains a heterologous DNA binding domain derived from a second nuclear hormone receptor, the cognate response element specifically binds the DNA binding domain. For example, where a RARα nuclear hormone receptor contains an estrogen receptor DNA binding domain fused to an RARα ligand binding domain, the cognate response element is an estrogen receptor response element (ERE). Similarly, where a RARα nuclear hormone receptor contains a GAL4 DNA binding domain fused to an RARα ligand binding domain, the cognate response element is a GAL4 binding site.

It is understood that a cognate response element can contain naturally occurring or synthetic nucleotides joined by phosphodiester linkages. In addition to common nucleotides such as adenosine 5′ phosphate, thymidine 5′ phosphate, cytidine 5′ phosphate, uracil 5′ phosphate and guanisine 5′ phosphate, a cognate response element can contain rare nucleotides such as hypoxanthine, xanthine, methylated, or methoxylated derivatives of common nucleotides, and the like. Such nucleotides may be ribonucleotides or deoxyribonucleotides, and the nucleic acids may be DNA, RNA or hybrids thereof. It further is understood that a cognate response element can be a synthetic nucleic acid molecule, for example, a peptide nucleic acid (PNA), or can contain 2′O-methylribonucleotides derivatives.

The following examples are intended to illustrate but not limit the present invention.

### EXAMPLE I

### A Dominant Negative RXRα Fails to Release Corepressor Despite Coactivator Recruitment

This example demonstrates that a dominant negative form of RXRα can simultaneously interact with corepressor and coactivator. This example further demonstrates that RAR can play a role in corepressor association while RXR can play a role in corepressor dissociation.

The dominant negative RXR derivative, RXRαΔC, which is truncated at amino acid 448 and lacks the C-terminal AF2 domain/helix 12, abrogates transcriptional activation of RAR ligands when expressed as a heterodimer with RAR (Feng et al., Genes Dev. 11:59-71 (1997)). The RAR “CoR box” is located within a region of the RAR ligand binding domain, helix 1, which organizes the tertiary structure of the remainder of the receptor in a ligand dependent manner, and has previously been demonstrated to be required for corepressor interaction. In view of studies indicating that nuclear receptor interaction domains for corepressors and coactivators may overlap (Horlein et al., Nature 277:397-404 (1995); Nagy et al., Genes Dev. 13:3209-3216 (1999); Hu and lazar, Nature 402:93-96 (1999)), coregulator interactions with RXRαΔC were analyzed alone and in combination with the RAR CoR box mutant RARα-V5-AHT, in the context of the RAR/RXR/DNA ternary complex.

As shown in FIG. 1, wild type RARα/RXRα heterodimer released the corepressor N-CoR and recruited the coactivator SRC-1 upon treatment with agonist TTNPB. FIG. 1 also shows the effect of the dominant negative RXRα, RXRαΔC, upon coregulator recruitment to the RAR/RXR/DNA ternary complex. Specifically, the RARα/RXRαΔC heterodimer failed to release N-CoR upon binding TTNPB (FIG. 1, lane 4, middle panel). In agreement with previous findings (Horlein et al., supra, 1995), N-CoR interaction with the RARα-AHT/RXRα heterodimer was significantly reduced relative to wild type RARU (FIG. 1, lanes 5 and 6). However, substitution of RXRαΔC for RXRα in the double mutant RARα-AHT/RXRΔC partially rescued N-CoR interaction (lanes 7 and 8), although the interaction with corepressor was not sensitive to agonist treatment. Analysis of coimmunoprecipitated RXRα and RXRαΔC indicated that comparable amounts of both forms of RXR were present in the RAR heterodimer (FIG. 1, bottom panel). Thus, these results indicate that RARα-AHT has a tertiary structure which is sufficient for RXR heterodimerization but which is unfavorable for N-CoR association.

Recruitment of the SRC-1 coactivator molecule also was analyzed. As shown in FIG. 1, deletion of the RXR AF2 domain resulted in a ternary complex which responded to agonist treatment by recruiting SRC-1 in spite of the failure to release corepressor (lane 4, top panel). Similar to the observed recruitment of corepressor N-CoR, agonist mediated SRC-1 interaction with the ternary complex containing RARα-AHT was mildly decreased compared to complex containing wild type RARα. In the double mutant ternary complex, SRC-1 recruitment was partially rescued. These data indicate that, in the context of the ternary complex, RAR can be involved in corepressor association while RXR can be involved in corepressor dissociation.

In sum, the coregulator interactions observed with RXRαΔC indicate that the dominant negative phenotype is not due to failure to recruit coactivator but, rather, failure to release corepressor. These results indicate that synthetic retinoid ligands can be designed which result in simultaneous coactivator and corepressor interaction at a ternary complex.

Cells were transfected and cell extracts prepared essentially as follows. For whole cell extracts, CV-1 cells were cultured with D-MEM (Gibco-BRL Life Technologies; Rockville, Md.) containing 10% activated charcoal extracted fetal bovine serum (Gemini Bio-Products). At a density of 40˜60% (15-cm plate, Falcon; Fort Worth, Tex.), cells were transiently transfected with 15 μl FuGene 6 Transfection Reagent (Roche Diagnostics) with 0.5 ug of pRS-RXRα, and 5 ug of either pcDNA3.1-hRARα-V5, pcDNA3.1-hRARβ-V5 or pcDNA3.1-hRARγ-V5 per plate. After two days, cells were rinsed twice with PBS and lysed in cold NET buffer (20 mM Tris-Cl [pH8.0], 200 mM NaCl, 1 mM EDTA, 0.1% NP-40, 10% glycerol) containing protease inhibitors, homogenized by QIAshredder (Qiagen; Valencia, Calif.), and clarified by centrifugation.

Immunoprecipitations and western analyses were performed as follows. CV-1 whole cell extract from transfected cells (1 mg) was used for each immunoprecipitation. Cell lysates were incubated with ligands on ice for 1 hour. Where indicated, annealed double-strand oligonucleotides (DR-5 RARE: 5′-AGCTTTCAGGTCACCAGGAGGTCAGAA-3′; SEQ ID NO: 17) were added prior to ligand addition and incubated on ice for 30 minutes. After a 1 hour incubation on ice with primary antibody (mouse anti-V5, Invitrogen), protein G-agarose (SIGMA) was added and samples were rocked overnight at 4° C. After washing with ice cold NET buffer, immunoprecipitants were resolved on SDS-polyacrylamide gels (4-12%) followed by western blotting. Membranes were probed with the indicated antibodies in PBS-T buffer (PBS with 0.1% Tween-20) containing 5% nonfat dry milk, and washed in PBS-T buffer. Primary antibodies (#SC553, rabbit anti-RXRα; #SC1609, goat anti-N-CoR) were obtained from Santa Cruz Biotech and Affinity BioReagents (#MA1-840, mouse anti-SRC-1; Golden, Colo.).

### EXAMPLE II

### A RARα Mutation Resulting in Corepressor Retention

This example demonstrates that a mutation near the CoR box of RARα mutant results in corepressor retention.

Arg₂₇₂ is located at the C-terminal end of helix 5 in close proximity to the CoR box in RARα. Using the ternary complex assay, coregulator interaction was analyzed with a RARα-V5 mutant in which alanine is substituted for arginine at position 272 (mutant R₂₇₂-A). As shown in FIG. 2A, TTNPB mediated SRC-1 recruitment was not altered in the RARα-V5 mutant R₂₇₂-A relative to SRC-1 recruitment by wild type RARα. However, N-CoR association with the unliganded ternary complex harboring this mutation was increased; moreover, dissociation upon addition of agonist was severely impaired (FIG. 2, top panel, compare lanes 2 and 4 to lanes 1 and 3, respectively). Thus, the RARα R₂₇₂-A mutation resulted in TTNPB induced coregulator recruitment similar to that seen for RARα/RXRαΔC and represents an additional example of agonist induced coactivator interaction with ternary complex in the absence of corepressor release.

### EXAMPLE III

### THE AF2 Domain of RAR is Required for Corepressor Interaction

This example demonstrates that helix 12 of activation domain 2 (AF2) is required for corepressor interaction with unliganded RARα.

Crystallographic studies comparing the ligand binding domains of apo-RXRα, holo-RARγ and PPARγ indicate that the helix 12 AF2 domain can be reoriented upon agonist binding, resulting in generation of a coactivator interaction domain. In the case of RARγ, juxtaposition of helix 12 alongside helices 3 and 4 is facilitated by formation of a salt bridge between Glu₄₁₄ and Lys₂₆₄. In addition, mutational analyses have demonstrated the requirement of Glu₄₁₄, Lys₂₆₄ and nearby residues for receptor transactivation.

Analogous mutations were generated in RARα-V5 and analyzed in the DNA dependent coregulator recruitment assay described above. Consistent with the previous demonstration that Glu₄₁₂ in the AF2 domain of RARα was required for ligand inducible transactivation, TTNPB mediated recruitment of SRC-1 to the ternary complex was significantly decreased with mutation of Glu₄₁₂ to Arg or Ala, or mutation of Lys₂₆₂ to Ala (FIG. 2B, lanes 2-4, middle panel). In contrast, as shown in FIG. 2A, lane 5, and described in Example II, mutation of arginine 272 to alanine did not significantly impair SRC-1 recruitment.

The RARα E₄₁₂-R, E₄₁₂-A and K₂₆₂-A mutants also exhibited decreased association with N-CoR as compared to unliganded wild type receptor (FIG. 2B, lanes 6-10, top panel). Specifically, mutation of either Glu₄₁₂ or Lys₂₆₂ in a manner which would perturb salt bridge formation between helix 12 and helix 4 led to a significant decrease in N-CoR association with the ternary complex in the absence of ligand. These results demonstrate that corepressor interaction requires specific interactions between helix 12 and helix 3 or 4 of RARα.

### EXAMPLE IV

### Differential N-CoR Dissociation by RARα Specific Ligands

This example demonstrates that RAR ligands with similar binding affinities can differentially effect corepressor recruitment and release.

Divergent amino acid residues within the ligand binding domains of different RAR isotypes provide a structural basis for the RARα subtype selectivity observed for synthetic retinoid ligands containing an internal amide linkage. RARα selective ligands shown in Table 2 share this amide linkage structure as well as similar affinities for RARα. However, in contrast to their similar binding affinities, these ligands exhibited disparate transactivation properties at RARα (see FIG. 3A). Specifically, AGN194365 exhibited potent and effective transactivation properties at RARα which were comparable to TTNPB. AGN194794 also activated RARα, albeit with slightly less efficacy and potency. In contrast, AGN196382 and 196412 had no activity at RARα except at the highest dose (1 μM) tested.

TABLE 2
Relative Kds for RARα

| TTNPB | RARα |
|---|---|
| 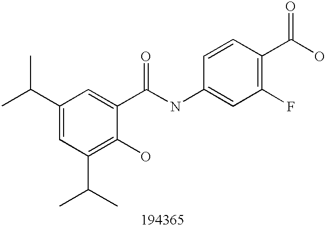 | 3 (37 LG) |
| 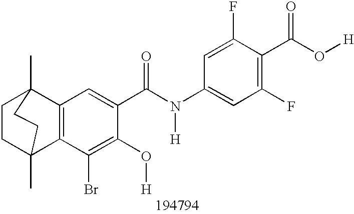 | 69, 99 |
| 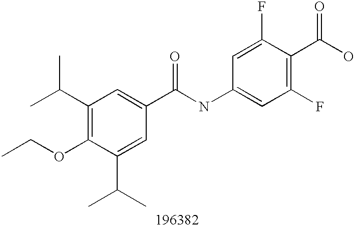 | 27 |
| 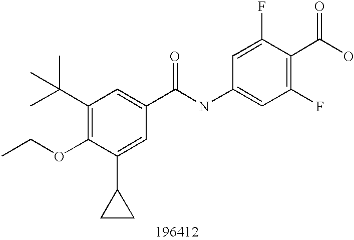 | 6 |

The DNA dependent coregulator recruitment assay was used to measure the ability of RARα selective ligands shown in Table 2 to recruit the coactivators SRC-1 and ACTR to the RARα/RXRα heterodimer bound to a DR-5 retinoic acid receptor response element (RARE). As expected, association of both coactivator proteins with the ternary complex required ligand (FIG. 3B). ACTR recruitment in response to the different ligands was relatively similar, with 194365, 194794 and 196382 resulting in 82-88% recruitment compared to TTNPB, and 196412 resulting in 65% recruitment relative to TTNPB. In contrast, the amount of SRC-1 recruitment was far more divergent in response to the different ligands. AGN194365 provided similar degree (65%) of SRC-1 recruitment compared to TTNPB, while SRC-1 recruitment by the remaining compounds was considerably weaker, ranging from 23 to 32% relative to TTNPB.

The amount of SRC-1 recruited to the ternary complex did not correlate with the transactivation profiles of the compounds. Specifically, the efficiency of SRC-1 recruitment by 194794, 196382 and 196412 was very similar yet AGN194794 was a significantly more efficient transactivator of RARα (FIG. 3A). The recruitment of coactivator p300 and N-CoR was also analyzed; p300 recruitment was nearly equivalent by 194794 and 196382 (34% and 30% relative to TTNPB, respectively). However, in contrast to dissociation of N-CoR by TTNPB, 194365 or 194794, ligand mediated dissociation of N-CoR in response to 196382 or 196412 was significantly impaired.

RAR transactivation assays were performed essentially as previously described. For analysis of ER-RARα chimeric receptor transactivation, 5×10³ CV-1 cells were plated per well of a 96-well microtiter plate and were transiently transfected with Lipofectamine and the indicated DNA. Specifically, transfections were performed with 0.1 μg of pERE-tk-Luc reporter, which contains the estrogen regulated element of the Xenopus vittelogenin A2 gene inserted into the plasmid tk-Luciferase, and 0.01 μg of the SV-40 based vector pECE carrying a chimeric ER-RAR receptor consisting of the estrogen receptor A/B and DNA binding domains fused to the DEF domain of RARα, β or γ. Alternatively, cells were cotransfected with the reporter plasmid MTV-4 (R5G)-Luciferase together with the plasmid pRS-hRXRα and either pRS-RARα-P-GR, pcDNA3-RARβ-P-GR, or pcDNA3-RARγ-P-GR as previously described (Klein et al., J. Biol. Chem. 271:22692-22696 (1996)). After eighteen hours, cells were rinsed with phosphate buffered saline (PBS) and fed with D-MEM (Gibco-BRL) containing 10% activated charcoal extracted fetal bovine serum (Gemini Bio-Products). Cells were treated with retinoids for 18 hours after which they were lysed; luciferase activity was measured as previously described, with luciferase values representing the mean±SEM of quadruplicate determinations.

These results indicate that SRC-1 recruitment does not necessarily correlate with transactivation activity and that transactivation activity can be correlated with the ability to recruit coactivator in combination with corepressor release.

### EXAMPLE V

### Analysis of Mechanism of Dominant Negative Receptor Activity

This example demonstrates that retention of N-CoR is not sufficient for dominant negative receptor activity.

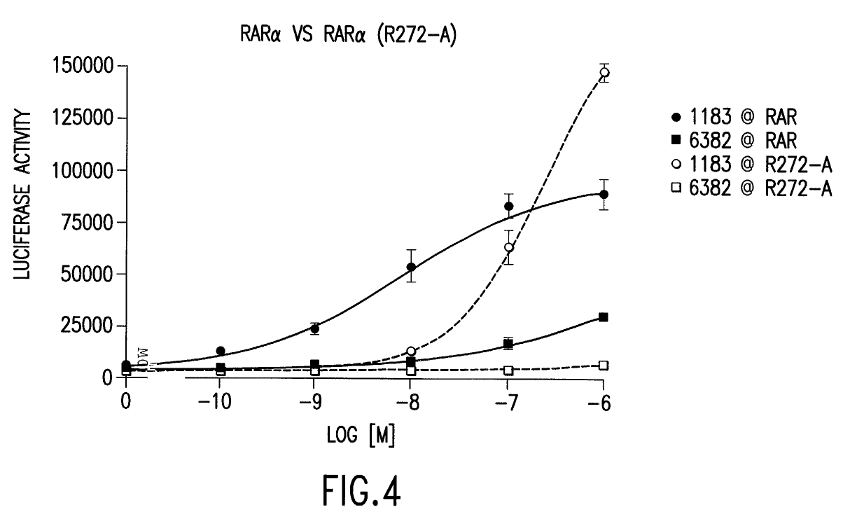

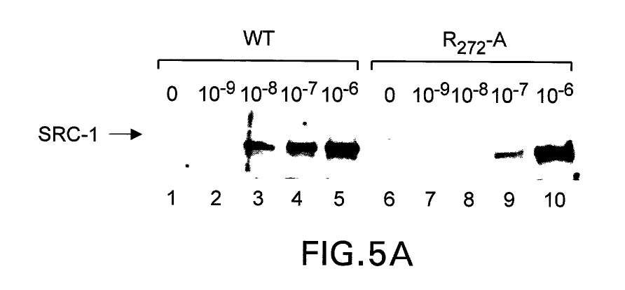

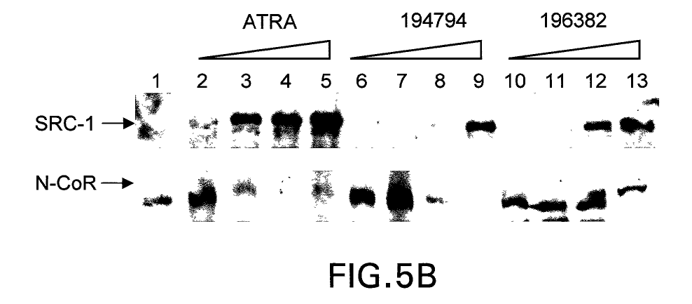

As disclosed above, agonist treatment of the ternary complex containing the mutant RARα(R₂₇₂-A) failed to release N-CoR (see FIGS. 2A and B). The RAR=60 R₂₇₂-A mutant was analyzed for dominant negative activity using a retinoid reporter system responsive to transfected RARs recognizing RAREs containing a glucocorticoid receptor half-site (Klein et al., supra, 1996). The responsiveness of the RARα mutant R₂₇₂-A to the RAR specific ligand TTNPB was compared to the responsiveness of wild type RARα. As shown in FIG. 4, TTNPB activation of the RARα(R₂₇₂-A) receptor was severely impaired with an EC₅₀ shifted greater than 1 log compared to wild type RARα. However, RARα(R272-A) activity consistently exceeded that of wild type RARα at the highest dose of TTNPB tested (1 μM). By comparison, activation of wild type RARα by AGN196382 in this system was significantly reduced relative to TTNPB, similar to that demonstrated in the chimeric ER-RARα assay (see FIG. 3A); RARα(R₂₇₂-A) was not activated at all. The latter result is consistent with the lack of recruitment of SRC-1 seen by AGN196382 in the RARα R₂₇₂-A containing ternary complex (see FIG. 2B). The reduced transactivation potency exhibited by TTNPB at RARα R₂₇₂-A was corroborated by a similar shift in potencies in the recruitment of SRC-1 (FIG. 5A) and the dissociation of N-CoR.

These results indicate that RARα R₂₇₂-A does not exhibit dominant negative activity but, rather, exhibits reduced affinity for the retinoid ligands tested. Furthermore, in comparison with wild type RARα, the increased interaction of RARα R₂₇₂-A with N-CoR at 1 μM TTNPB, (see FIG. 2A) was not sufficient to antagonize transcriptional activity resulting from coactivator recruitment.

In contrast to RARα R₂₇₂-A mutant coregulator interactions, interactions with the wild type RAR/RXR/RARE ternary complex in response to escalating doses of AGN194794 or AGN196382 indicated that the weaker transcriptional activity mediated by the latter was not the result of a difference in SRC-1 recruitment between these two ligands (FIG. 5B). Specifically, the efficacy and potency for SRC-1 recruitment was essentially identical for the two ligands. In contrast, N-CoR dissociation in response to the binding of AGN196382 was nearly absent while AGN194794 binding resulted in full N-CoR disengagement.

### EXAMPLE VI

### Anti-AP1 Activity of Dissociated RAR Ligands

This example demonstrates that the retinoid ligand, AGN196382, exhibits anti-AP1 activity while only exhibiting very weak RARα transcriptional activity.

The ability of AGN196382 to antagonize phorbol ester stimulated transcription was assayed using a collagenase-promoter reporter construct containing an AP-1 response element. As previously demonstrated, and consistent with avid recruitment of coactivator molecules such as SRC-1, the synthetic RAR agonist TTNPB exhibited potent and efficacious anti-AP-1 activity (see FIG. 6). Furthermore, AGN196382 treatment was similarly efficacious in antagonizing TPA stimulated collagenase reporter activity. However, the potency of anti-AP-1 activity mediated by 196382 was considerably reduced relative to TTNPB, consistent with apparent differences in mediating SRC-1 recruitment (compare FIGS. 5A and 5B). Specifically, detection of anti-AP-1 activity by AGN196382 was only detectable at doses greater than 10 μM; similarly, detection of SRC-1 recruitment to the ternary complex by 196382 was apparent only at doses greater than 10 μM. Such elevated doses of AGN196382, while sufficient for efficacious anti-AP-1 activity, are not sufficient for efficient transcriptional activation of RARα.

These results demonstrate that hormone ligands can have selective indirect effects on other signaling pathways, such as anti-AP-1 activity, even in the absence of direct transcriptional effects mediated by the hormone receptor. These results further indicate that such dissociated ligands can be identified based on their ability to induce coactivator recruitment along with corepressor retention.

All journal article, reference and patent citations provided above, in parentheses or otherwise, whether previously stated or not, are incorporated herein by reference in their entirety.

Although the invention has been described with reference to the examples provided above, it should be understood that various modifications can be made without departing from the spirit of the invention. Accordingly, the invention is limited only by the claims.

We claim:

1. A method of identifying an effective agent that dissociates nuclear hormone receptor activities, comprising the steps of:
(a) contacting a nuclear hormone receptor with one or more agents under conditions suitable for forming a test complex comprising nuclear hormone receptor dimer, coactivator and corepressor;
(b) assaying for coactivator association with said test complex; and
(c) assaying for corepressor association with said test complex,
wherein coactivator association combined with corepressor association indicates that at least one of said agents is an effective agent that dissociates nuclear hormone receptor activities.

2. A method of identifying an effective agent that dissociates nuclear hormone receptor activities, comprising the steps of:
(a) contacting a nuclear hormone receptor with one or more agents under conditions suitable for forming a ternary complex comprising nuclear hormone receptor dimer, bound cognate response element, coactivator and corepressor;
(b) assaying for coactivator association with said ternary complex; and
(c) assaying for corepressor association with said ternary complex,
wherein coactivator association combined with corepressor association indicates that at least one of said agents is an effective agent that dissociates nuclear hormone receptor activities.

3. The method of claim 1, wherein said contacting is performed in vitro.

4. The method of claim 1, wherein said nuclear hormone receptor is contacted with said one or more agents in the presence of a eukaryotic cell sample.

5. The method of claim 4, wherein said eukaryotic cell sample comprises viable cells.

6. The method of claim 4, wherein said eukaryotic cell sample comprises a whole cell lysate.

7. The method of claim 4, wherein said eukaryotic cell sample comprises a fractionated cell lysate.

8. The method of claim 4, wherein said eukaryotic cell sample comprises an exogenous nucleic acid molecule encoding said nuclear hormone receptor.

9. The method of claim 4, wherein said coactivator is endogenous to said cell.

10. The method of claim 4, wherein said corepressor is endogenous to said cell.

11. A method of identifying an effective agent that dissociates nuclear hormone receptor activities, comprising the steps of:
(a) contacting a nuclear hormone receptor with one or more agents under conditions suitable for forming a test complex comprising nuclear hormone receptor dimer, coactivator and corepressor,
wherein said nuclear hormone receptor is selected from the group consisting of a retinoic acid receptor, retinoid X receptor, thyroid receptor, estrogen receptor and peroxisome proliferator activated receptor;
(b) assaying for coactivator association with said test complex; and
(c) assaying for corepressor association with said test complex,
wherein coactivator association combined with corepressor association indicates that at least one of said agents is an effective agent that dissociates nuclear hormone receptor activities.

12. The method of claim 11, wherein said nuclear hormone receptor is selected from the group consisting of RARα, RARβ, RARγ, RXRα, RXRβ and RXRγ.

13. The method of claim 12, wherein said nuclear hormone receptor is a retinoic acid receptor selected from the group consisting of RARα, RARβ and RARγ.

14. The method of claim 1, wherein said coactivator is selected from the group consisting of
SRC-1/NCoA-1;
TIF-2/GRIP-1/NCoA-2;
ACTR/p/CIP/AIB1/NCoA-3;
p300/CBP;
p/CAF; and
TATA box binding protein.

15. The method of claim 14, wherein said coactivator is SRC-1/NCoA-1.

16. The method of claim 1, wherein said corepressor is selected from the group consisting of N-CoR and SMRT.

17. The method of claim 16, wherein said corepressor is N-CoR.

18. A method of identifying an effective agent that dissociates nuclear hormone receptor activities, comprising the steps of:
(a) contacting a nuclear hormone receptor with one or more agents under conditions suitable for forming a test complex comprising nuclear hormone receptor dimer, coactivator and corepressor;
(b) assaying for coactivator association with said test complex, wherein said coactivator is selected from the group consisting of SRC-1/NCoA-1, TIF-2/GRIP-1/NCoA-2, ACTR/p/CIP/AIB1/NCoA-3, p300/CBP, p/CAF, and TATA box binding protein (TBP); and
(c) assaying for corepressor association with said test complex, wherein said corepressor is selected from the group consisting of N-CoR and SMRT,
wherein coactivator association combined with corepressor association indicates that at least one of said agents is an effective agent that dissociates nuclear hormone receptor activities.

19. The method of claim 1, wherein step (b) comprises specific binding to said test complex.

20. The method of claim 19, wherein step (b) comprises immunoprecipitation of said test complex.

21. The method of claim 20, wherein said immunoprecipitation is performed using antibody immunoreactive with said nuclear hormone receptor dimer.

22. The method of claim 19, wherein step (b) comprises immunodetection of said coactivator.

23. The method of claim 1, wherein step (c) comprises specific binding to said test complex.

24. The method of claim 23, wherein step (c) comprises immunoprecipitation of said test complex.

25. The method of claim 24, wherein said immunoprecipitation is performed using antibody immunoreactive with said nuclear hormone receptor dimer.

26. The method of claim 23, wherein step (c) comprises immunodetection of said corepressor.
